(19) United States
(12) Patent Application Publication
(10) Pub. No.: US 2014/0114067 A1
(43) Pub. Date: Apr. 24, 2014

(54) BENZIMIDAZOLE DERIVATIVES AS MITOCHONDRIAL FUNCTION MODULATORS

(71) Applicant: KOREA INSTITUTE OF SCIENCE AND TECHNOLOGY, Seoul (KR)

(72) Inventors: Ae Nim PAE, Seoul (KR); Sun Joon MIN, Seoul (KR); Eun Joo ROH, Seoul (KR); Ha Yun YANG, Goyang (KR); Tae Hoon KIM, Suwon (KR); Beoung Gun PARK, Donghae (KR); Yong Seo CHO, Seoul (KR)

(73) Assignee: KOREA INSTITUTE OF SCIENCE AND TECHNOLOGY, Seoul (KR)

(21) Appl. No.: 14/048,979

(22) Filed: Oct. 8, 2013

(30) Foreign Application Priority Data
Oct. 22, 2012 (KR) 10-2012-0117057

(51) Int. Cl. C07D 413/06; C07D 403/06; C07D 401/06; C07D 233/26

(52) U.S. Cl. CPC C07D 413/06; C07D 233/26; C07D 403/06; C07D 401/06
USPC 544/139; 548/309.7; 548/306.1; 546/199; 544/370

(57) ABSTRACT

Provided are a benzimidazole derivative modulating mitochondrial functions and having pharmaceutical activity as a neuro-protective agent, and a pharmaceutical composition including the compound as an active ingredient.

### CROSS-REFERENCE TO RELATED APPLICATIONS

This application claims priority under 35 U.S.C. §119 to Korean Patent Application No. 10-2012-0117057, filed on Oct. 22, 2012, in the Korean Intellectual Property Office, the disclosure of which is incorporated herein by reference in its entirety.

### TECHNICAL FIELD

The present disclosure relates to benzimidazole derivatives that modulate mitochondrial functions and show pharmaceutical activities as neuro-protective agents, a method for preparing the same, and a pharmaceutical composition including the same as active ingredients.

### BACKGROUND

Mitochondria are organelles that produce adenosine triphosphate (ATP) through oxidative phosphorylation, and are main energy sources for maintaining normal functions of brain cells [Sluse Fe, Acta Biochim Polon 1996, 43, 349-360]. Thus, mitochondria play an important role in apoptosis as well as in energy generation in cells. Functional deformation of such mitochondria becomes a direct or indirect cause of degenerative brain disorders, such as Alzheimer's disease, diabetes, Parkinson's disease, ischemic brain diseases (e.g., stroke), Huntington's disease, schizophrenia, etc, [Schapira A H, Lancet 2006, 368(9529), 70-82; Lim Y A et al., Proteomics 2010, 10(8), 1621-33; Pieczenik et al., Exp. Mol. Pathol. 2007, 83(1), 84-92].

It is reported that mitochondrial function disorders reduce ATP production, increase production of active oxygen (super oxide, peroxynitrite, hydroxyl radicals, hydrogen peroxide, etc.), break calcium homeostasis, and deliberate apoptosis factors [Sugioka et al., Biochim Biophys Acta 1988, 936, 377-385; Martinou et al., Nature cell Biol 2000, 2, E41-E43]. Lipid peroxidation caused by active oxygen is known as a main cause of injury to central nervous system found in degenerative diseases and ischemic diseases. In addition, it is known that mitochondrial function disorders play an important role in apoptosis occurring in various cells [Kroemer et al., FASEB J. 1995, 9, 1277-87]. In various cells including neurons, symptoms of a drop in mitochondrial membrane potential (DYm), i.e., those of mitochondrial function disorders cause apoptosis through decomposition of nuclear DNA [Lill and Neupert, Trends Cell Biol 1996, 6, 56-61; Newmeyer et al., Cell 1994, 70, 353-64].

Alzheimer's disease is a typical example of degenerative diseases of central nervous system. Alzheimer's dementia is a multifactorial disease and the mechanism thereof is not known clearly. However, it is characterized by neuronal damage caused by amyloid beta (Ab) [Lustbader et al., Science 2004, 304(5669), 448-52]. Introduction of amyloid beta clusters into cells causes interruption of protein signals and breakage of calcium ion homeostasis. In addition, Ab is introduced into mitochondria to form complexes with cyclophilin D, resulting in opening of mitochondrial permeability transition pores [Heng et al., Biochimica et Biophysica acta 2010, 1802, 198-204]. Necrosis occurring excessively in brain cells through such a process causes Alzheimer's disease [Anatoly and Flint, Nature medicine 2008, 14(10), 1020-1021]. Therefore, when interrupting a peripheral benzodiazepine receptor which is a constituent of mitochondrial permeability transition pores, it is possible to inhibit the opening of mitochondrial permeability transition pores, and thus to inhibit necrosis [Rainer et al., Nature reviews 2010, 9, 971-988; W. Kugler et al., Cellular oncology 2008, 30, 435-450]. There have not been developed methods of preventing or treating mitochondrial function disorders.

Accordingly, the present inventors have developed a novel therapeutic agent capable of recovering mitochondrial functions damaged by amyloid beta (Ab).

### SUMMARY

An embodiment of the present invention is directed to providing a novel benzimidazole derivative and pharmaceutically acceptable salt thereof.

Another embodiment of the present invention is directed to providing a method for preparing the novel benzimidazole compound, which includes forming benzimidazole scaffolds in the presence of various catalysts, forming intermediates through alkyl substitution reaction, and subjecting acids obtained by hydrolysis to condensation with aniline or alkylamine having various substituents.

Still another embodiment of the present invention is directed to providing a pharmaceutical composition including, as an active ingredient, the above-mentioned benzimidazole derivative and pharmaceutically acceptable salt thereof, and effective for recovering mitochondrial functions damaged by amyloid beta.

Yet another embodiment of the present invention is directed to providing a medicament for preventing and treating diseases, including Alzheimer's disease, Parkinson's disease, Huntington's disease, ischemic brain diseases (e.g., stroke), diabetes and schizophrenia, wherein the medicament includes, as an active ingredient, the above-mentioned benzimidazole derivative and pharmaceutically acceptable salt thereof,

In one general aspect, there is provided a benzimidazole derivative represented by the following Formula 1, a method for preparing the same, and pharmaceutical use of the benzimidazole derivative.

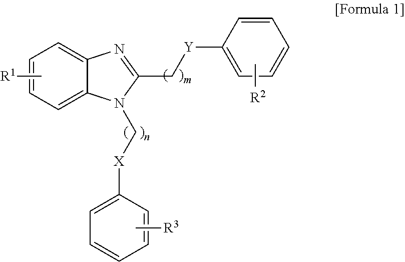

wherein X and Y are the same or different from each other and each represents a single bond or thiomethylene group, —C(O)NH— or —NHC(O); R¹ represents a hydrogen atom or halogen atom; R² and R³ are the same or different from each other and each represents a hydrogen atom, halogen atom, C₁-C₆ alkyl group, C₁-C₆ alkoxy group, phenoxy group, biphenyloxy group or —C(O)NR⁴R⁵; R⁴ and R⁵ are the same or different from each other and each represents a hydrogen atom, C₁-C₆ alkyl group or —(CH₂)l-NR⁶R⁷ (wherein R⁶ and R⁷ are the same or different from each other and each represents a hydrogen atom or C₁-C₆ alkyl group, or R⁶ and R⁷ represent a heteroalicyclic group selected from pyrrolidinyl, piperidinyl, piperazinyl and morpholino formed when they are bound to each other, and the heteroalicyclic group is substituted or non-substituted with a C₁-C₆ alkyl group); and l, m and n represent an integer of 0 to 6, with the proviso that when X and Y represent single bonds, R³ represents —C(O)NH—(CH₂)ₗ—NR⁶R⁷ (wherein R⁶ and R⁷ are bound to each other to form a heteroaliphatic cyclic group selected from the group consisting of pyrrolidinyl, piperidinyl, piperazinyl and morpholino, and the heteroaliphatic cyclic group is substituted or non-substitute with C₁-C₆ alkyl).

### DETAILED DESCRIPTION OF EMBODIMENTS

The advantages, features and aspects of the present invention will become apparent from the following description of the embodiments with reference to the accompanying drawings, which is set forth hereinafter.

In one aspect, there is provided a benzimidazole derivative represented by the above Formula 1. The benzimidazole derivative represented by the above Formula 1 may form a pharmaceutically acceptable salt by a method generally known in the art. Such a pharmaceutically acceptable salt should have low toxicity to the human body and have no adverse effect upon the biological activity and physicochemical properties of its parent compound. Pharmaceutically acceptable salts may include acid addition salts formed between pharmaceutically acceptable free acids and the alkaline compound of Formula 1, alkali metal salts (e.g., sodium salt), alkaline earth metal salts (e.g., calcium salt), organic base addition salts formed between organic bases and the carboxylic acid of Formula 1, and aminoacid addition salts. Such free acids that may be used for preparing pharmaceutically acceptable salts may be classified into inorganic salts and organic salts. Inorganic acids that may be used include hydrochloric acid, sulfuric acid, nitric acid, phosphoric acid, hyperchloric acid, hydrobromic acid, or the like. Organic acids that may be used include acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid, glutamic acid, or the like. Organic bases that may be used for preparing organic base addition salts include tris(hydroxymethyl)methylamine, dicyclohexylamine, or the like. Aminoacids that may be used for preparing amino acid addition salts include natural amino acids, such as alanine, glycine, or the like.

In addition to the above-described pharmaceutically acceptable salts, the benzimidazole derivative represented by Formula 1 also includes any hydrate and solvate. The pharmaceutically acceptable salts may be obtained by the general method as described hereinafter. The compound of Formula 1 in its base form is dissolved into a water-miscible solvent such as methanol, ethanol, acetone or 1,4-dioxane, and a free acid or free base is added thereto, followed by crystallization or recrystallization.

In addition, the benzimidazole derivative represented by Formula 1 may have at least one asymmetric center. In this case, the benzimidazole derivative may have isomers or diastereomers. Therefore, the present invention includes each isomer or a mixture of such isomers. Different isomers may be separated or isolated from each other by a conventional method. Otherwise, any desired isomer may be obtained by a conventional method, or by stereospecific or asymmetric synthesis.

Further, the present invention includes a radioactive derivative of the compound represented by Formula 1. Such a radioactive derivative is useful for biotrepy.

Hereinafter, the substituents used for defining the benzimidazole derivative represented by Formula 1 will be explained in detail.

As used herein, ‘alkyl group’ includes any linear, branched and cyclic carbon chains having 1 to 6 carbon atoms, and preferred alkyl groups include methyl, ethyl, n-propyl, isopropyl, n-butyl, isobutyl, tert-butyl, cyclopentyl, cyclohexyl group, or the like.

As used herein, ‘alkoxy group’ means a carbonaceous alkyl group linked to oxygen atom, wherein alkyl is the same as defined above, and preferred alkoxy groups include methoxy, ethoxy, n-propoxy, isopropoxy, n-butoxy, tert-butoxy group, or the like.

As used herein, ‘heteroalicyclic group’ means a 5- to 7-membered aliphatic heterocarbon cyclic group containing at least one hetero atom selected from nitrogen and oxygen, and preferred heteroalicyclic groups include pyrrolidine, piperidine, piperazine, morpholine, or the like. The heteroalicyclic group may be substituted or non-substituted with an alkyl substituent.

In the benzimidazole derivative represented by Formula 1, preferably, X and Y are the same or different from each other and each represents a single bond, thiomethylene group, —C(O)NH— or —NHC(O)—; R¹ represents a hydrogen atom or chloro; R² represents a hydrogen atom, chloro, methyl, ethyl, isopropyl, or methoxy group; R³ represents chloro, fluoro, methyl, isopropyl, tert-butyl, biphenyloxy, aminocarbonyl, (methylamino)carbonyl, (dimethylamino)carbonyl, N-(aminopropyl)amide, N-[3-(methylamino)propyl]amide, N-[3-(dimethylamino)propyl]amide, N-[3-(pyrrolidine-1-yl)propyl]amide, N-[1-(ethylpyrrolidin-2-yl)methyl]amide, N-[2-(piperidien-1-yl)ethyl]amide, N-[3-(piperidine-1-yl)propyl]amide, N-(1-methylpiperidin-4-yl)amide, N-[3-(4-methylpiperazin-1-yl)propyl]amide, or N-(3-morpholinopropyl)amide group; and m and n are the same or different from each other, and each represents an integer of 0, 1 or 2.

In the benzimidazole derivative represented by Formula 1, when X and Y represent single bonds, R³ preferably represents N-[3-(pyrrolidin-1-yl)propyl]amide group, N-[1-(ethylpyrrolidin-2-yl)methyl]amide group, N-[2-(piperidin-1-yl)ethyl]amide group, N-[3-(piperidin-1-yl)propyl]amide group, N-(1-methylpiperidin-4-yl)amide group, N-[3-(4-methylpiperazin-1-yl)propyl]amide group, or N-(3-morpholinopropyl)amide group.

In the benzimidazole derivative represented by Formula 1, preferred benzimidazole compounds may include those represented by the following Formulae 1a, 1b, 1c and 1d.

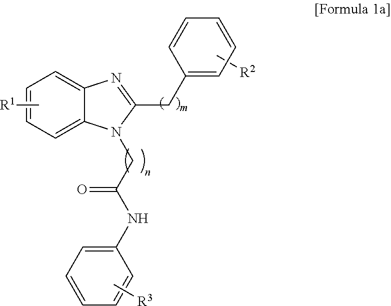

(wherein each of R¹, R², R³, m and n is the same as defined above).

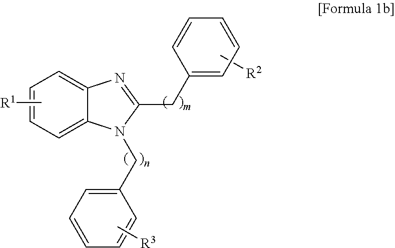

(wherein each of R¹, R², m and n is the same as defined above, and R³ represents —C(O)NH—(CH₂)ₗ—NR⁶R⁷ (wherein R⁶ and R⁷ are bound to each other to form a heteroaliphatic cyclic group selected from the group consisting of pyrrolidinyl, piperidinyl, piperazinyl and morpholino, and the heteroaliphatic cyclic group is substituted or non-substitute with C₁-C₆ alkyl)).

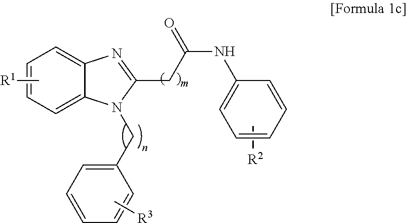

(wherein each of R¹, R², R³, m and n is the same as defined above).

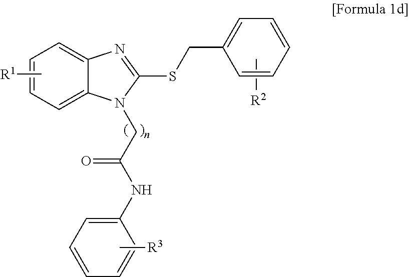

(wherein each of R¹, R², R³, m and n is the same as defined above).

Particular examples of the benzimidazole compound represented by Formula 1 include the following compounds:
Compound 1: 2-(2-(2,5-dimethoxyphenyl)-1H-benzo[d]imidazol-1-yl)-N-(5-isopropyl-2-methylphenyl)acetamide
Compound 2: N-(5-(biphenyl-4-yloxy)-2-fluorophenyl)-2-(2-(2,5-dimethoxyphenyl)-1H-benzo[d]imidazol-1-yl)acetamide
Compound 3: N-(3,5-dichlorophenyl)-2-(2-(2,5-dimethoxyphenyl)-1H-benzo[d]imidazol-1-yl)acetamide
Compound 4: N-(5-isopropyl-2-methylphenyl)-(2-phenyl-1H-benzo[d]imidazol-1-yl)acetamide
Compound 5: N-(3,5-dichlorophenyl)-2-(2-(2,5-dichlorophenyl)-1H-benzo[d]imidazol-1-yl)acetamide
Compound 6: N-(3,5-di-tert-butylphenyl)-2-(2-(2,5-dichlorophenyl)-1H-benzo[d]imidazol-1-yl)acetamide
Compound 7: N-(2-tert-butyl-6-methylphenyl)-2-(2-(2,5-dichlorophenyl)-1H-benzo[d]imidazol-1-yl)acetamide
Compound 8: 2-(2-benzyl-1H-benzo[d]imidazol-1-yl)-N-(5-isopropyl-2-methylphenyl)acetamide
Compound 9: 2-(2-benzyl-1H-benzo[d]imidazol-1-yl)-N-(5-biphenyl-4-yloxy)-2-fluorophenyl)acetamide
Compound 10: 2-(2-benzyl-1H-benzo[d]imidazol-1-yl)-N-(3,5-dichloro phenyl)acetamide
Compound 11: 2-(2-benzyl-1H-benzo[d]imidazol-1-yl)-N-(3,5-di-tert-butylpenyl)acetamide
Compound 12: 2-(2-benzyl-1H-benzo[d]imidazol-1-yl)-N-(2-tert-butyl-6-methylphenyl)acetamide
Compound 13: N-(3,5-di-tert-butylphenyl)-2-(2-phenyl-1H-benzo[d]imidazol-1-yl)acetamide
Compound 14: N-(5-isopropyl-2-methylphenyl)-2-(2-phenyl-1H-benzo[d]imidazol-1-yl)acetamide
Compound 15: N-(3,5-dichlorophenyl)-2-(2-phenyl-1H-benzo[d]imidazol-1-yl)acetamide
Compound 16: N-(2-tert-butyl-6-methylphenyl)-2-(2-phenyl-1H-benzo[d]imidazol-1-yl)acetamide
Compound 17: N-(3,5-di-tert-butylphenyl)-2-(2-(2,5-dimethoxybenzyl)-1H-benzo[d]imidazol-1-yl)acetamide
Compound 18: N-(3,5-dichlorophenyl)-2-(2-(2,5-dimethoxybenzyl)-1H-benzo[d]imidazol-1-yl)acetamide
Compound 19: 2-(2-(2,5-dimethoxybenzyl)-1H-benzo[d]imidazol-1-yl)-N-(5-isopropyl-2-methylphenyl)acetamide
Compound 20: N-(3,5-di-tert-butylphenyl)-2-(2-(2,6-dichlorobenzyl)-1H-benzo[d]imidazol-1-yl)acetamide
Compound 21: 2-(2-(3,4-dichlorobenzyl)-1H-benzo[d]imidazol-1-yl)-N-(5-isopropyl-2-methylphenyl)acetamide
Compound 22: 2-(5-chloro-2-(2,6-dichlorobenzyl)-1H-benzo[d]imidazol-1-yl)-N-(5-isopropyl-2-methylphenyl)acetamide
Compound 23: 2-(2-(2,6-dichlorobenzyl)-1H-benzo[d]imidazol-1-yl)-N-(5-isopropyl-2-methylphenyl)acetamide
Compound 24: N-(3,5-di-tert-butylphenyl)-2-(2-(3,4-dichlorobenzyl)-1H-benzo[d]imidazol-1-yl)acetamide
Compound 25: 2-(6-chloro-2-(2,5-dichlorophenyl)-1H-benzo[d]imidazol-1-yl)-N-(5-isopropyl-2-methylphenyl)acetamide
Compound 26: 2-(5-chloro-2-(2,5-dichlorophenyl)-1H-benzo[d]imidazol-1-yl)-N-(5-isopropyl-2-methylphenyl)acetamide
Compound 27: 2-(5-chloro-2-(2,5-dichlorophenyl)-1H-benzo[d]imidazol-1-yl)-N-(3,5-dichlorophenyl)acetamide
Compound 28: 2-(6-chloro-2-(2,5-dichlorophenyl)-1H-benzo[d]imidazol-1-yl)-N-(3,5-dichlorophenyl)acetamide
Compound 29: 2-(2-(3,4-dichlorobenzyl)-1H-benzo[d]imidazol-1-yl)-N-(3,5-dichlorophenyl)acetamide
Compound 30: 2-(6-chloro-2-(2,5-dichlorophenyl)-1H-benzo[d]imidazol-1-yl)-N-(3,5-di-tert-butylphenyl)acetamide
Compound 31: 2-(5-chloro-2-(2,5-dichlorophenyl)-1H-benzo[d]imidazol-1-yl)-N-(3,5-di-tert-butylphenyl)acetamide
Compound 32: 4-((2-(2,6-dichlorobenzyl)-1H-benzo[d]imidazol-1-yl)methyl)-N-((1-ethylpyrrolidin-2-yl)methyl)benzamide
Compound 33: 4-((2-(2,6-dichlorobenzyl)-1H-benzo[d]imidazol-1-yl)methyl)-N-(1-methylpiperidin-4-yl)benzamide
Compound 34: 4-((2-(2,6-dichlorobenzyl)-1H-benzo[d]imidazol-1-yl)methyl)-N-(3-(dimethylamino)propyl)benzamide
Compound 35: 4-((2-(2,6-dichlorobenzyl)-1H-benzo[d]imidazol-1-yl)methyl)-N-(3-(pyrrolidin-1-yl)propyl)benzamide
Compound 36: 4-((2-(2,6-dichlorobenzyl)-1H-benzo[d]imidazol-1-yl)methyl)-N-(3-(4-methylpiperazin-1-yl)propyl)benzamide
Compound 37: 4-((2-(2,6-dichlorobenzyl)-1H-benzo[d]imidazol-1-yl)methyl)-N-(3-(piperidin-1-yl)propyl)benzamide
Compound 38: 4-((2-(2,6-dichlorobenzyl)-1H-benzo[d]imidazol-1-yl)methyl)-N-(3-(piperidin-1-yl)ethyl)benzamide
Compound 39: 4-((2-(2,6-dichlorobenzyl)-1H-benzo[d]imidazol-1-yl)methyl)-N-(3-morpholinopropyl)benzamide
Compound 40: 2-(6-chloro-2-(2,6-dichlorobenzyl)-1H-benzo[d]imidazol-1-yl)-N-(5-isopropyl-2-methylphenyl)acetamide
Compound 41: N-((1-ethylpyrrolidin-2-yl)methyl)-4-((2-(2-(5-isopropyl-2-methylphenylamino)-2-oxoethyl)-1H-benzo[d]imidazol-1-yl)methyl)benzamide
Compound 42: N-(3-(dimethylamino)propyl)-4-((2-(2-(5-isopropyl-2-methylphenylamino)2-oxoethyl)-1H-benzo[d]imidazol-1-yl)methyl)benzamide
Compound 43: N-((1-ethylpyrrolidin-2-yl)methyl)-4-((1-(2-(5-isopropyl-2-methylphenylamino)₂-oxoethyl)-1H-benzo[d]imidazol-2-ylthio)methyl)benzamide
Compound 44: 4-((2-(2-(5-isopropyl-2-methylphenylamino)-2-oxoethyl)-1H-benzo[d]imidazol-1-yl)methyl)-N-(3-piperidin-1-yl)propyl)benz amide
Compound 45: N-(3-(dimethylamino)propyl)-4-((1-(2-(5-isopropyl-2-methylphenylamino)-2-oxoethyl)-1H-benzo[d]imidazol-2-ylthio)methyl)benzamide
Compound 46: 4-((1-(2-(5-isopropyl-2-methylphenylamino)-2-oxoethyl)-1H-benzo[d]imidazol-2-ylthio)methyl)-N-(3-piperidin-1-yl)propyl)benzamide
Compound 47: 4-((2-(2,6-dichlorobenzyl)-1H-benzo[d]imidazol-1-yl)methyl)-N,N-dimethylbenzamide

Meanwhile, the present invention also provides a method for preparing the benzimidazole compound represented by Formula 1. The method will be explained in detail hereinafter.

The compound represented by Formula 1 wherein X is —C(O)NH— may be obtained by the method as depicted in the following Reaction Scheme 1:

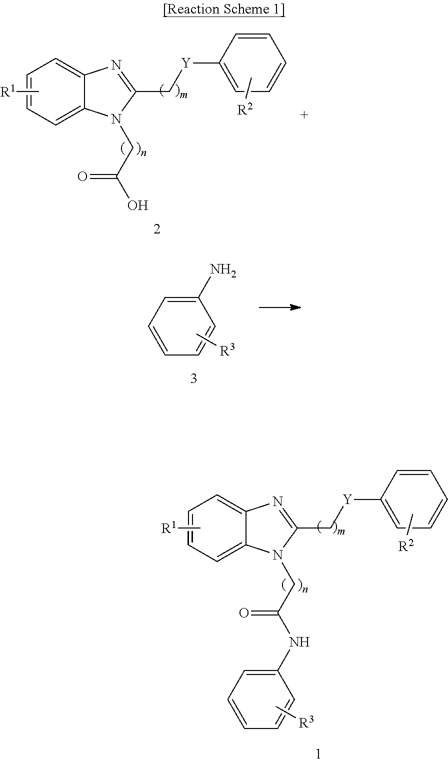

(wherein each of R¹, R², R³, Y, m and n is the same as defined in Formula 1).

According to Reaction Scheme 1, the acid compound represented by Formula 2 and the amine compound having various substituents and represented by Formula 3 may be subjected to condensation to obtain a benzimidazole compound represented by Formula 1 wherein X is —C(O)NH—.

The amidating agent that may be used for the condensation as depicted in Reaction Scheme 1 may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluoro phosphate (HBTU), (benzotriazol-1-yloxy)tripyrrolidinophosphonium hexafluorophosphate (pyBOP), (benzotriazol-1-yloxy)tris(dimethylamino) phosphonium hexafluorophosphate (BOP), or the like. In some embodiments of the present invention, HATU and BOP are used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) and diisopropylethylamine (DIPEA) are used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethylformamide is used mainly as a reaction solvent. The condensation temperature is preferably maintained within a range of 25° C. to 80° C. In some embodiments of the present invention, the condensation is carried out mainly at room temperature. The condensation may be carried out for 6-24 hours, preferably for 12-16 hours. After completing the condensation, the reaction mixture is concentrated under reduced pressure, washed with water, and subjected to extraction with a suitable organic solvent. Particular examples of the extraction solvent include ether, methylene chloride, chloroform and ethyl acetate, and more suitably ethyl acetate. After the extraction, the reaction mixture is purified through chromatography to isolate a pure product.

In addition, the acid compound used as a starting material for the method according to Reaction Scheme 1 may be obtained by carrying out the method as depicted in the following Reaction Scheme 2.

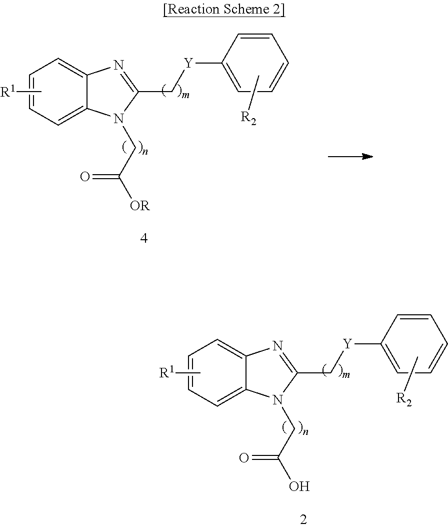

(wherein each of R¹, R², Y, m and n is the same as defined in Formula 1).

According to Reaction Scheme 2, the acid compound represented by the above Formula 2 may be obtained by hydrolysis of the ester compound represented by Formula 4.

Particular examples of the base that may be used in the hydrolysis according to Reaction Scheme 2 include potassium carbonate, sodium carbonate, lithium hydroxide, sodium hydroxide, potassium hydroxide, or the like. In some embodiments of the present invention, lithium hydroxide and sodium hydroxide are used mainly as a base. As the reaction solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, methanol and tetrahydrofuran are used mainly as a reaction solvent. The hydrolysis temperature is maintained preferably within a range of 25° C. to 80° C. In some embodiments of the present invention, the hydrolysis is carried out mainly at room temperature. The hydrolysis may be carried out for 30 minutes to 24 hours, preferably for 30 minutes to 4 hours. After completing the hydrolysis, the reaction mixture is concentrated under reduced pressure, dissolved in water and acidified with 1N hydrochloric acid. The resultant solid may be obtained by filtering without additional purification process.

In addition, the ester compound used as a starting material for the method according to Reaction Scheme 2 may be obtained by carrying out the method as depicted in the following Reaction Scheme 3.

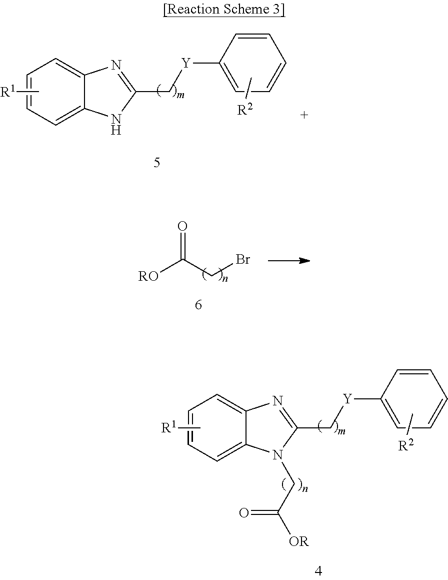

(wherein each of R¹, R², Y, m and n is the same as defined in Formula 1, and R is a C₁-C₆ alkyl group).

According to Reaction Scheme 3, the ester compound represented by Formula 4 may be obtained by carrying out alkylation between the imidazole compound represented by the above Formula 5 and alkyl 2-bromoalkyl carbonate represented by the above Formula 6.

Particular examples of the base that may be used in the alkylation according to Reaction Scheme 3 include potassium carbonate, sodium carbonate, lithium hydroxide, sodium hydroxide, potassium hydroxide, sodium hydride or the like. In some embodiments of the present invention, sodium hydride is used mainly as a base. As the alkylation solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethyl formamide is used mainly as a reaction solvent. The condensation temperature is maintained preferably within a range of 25° C. to 80° C. In some embodiments of the present invention, the reaction is carried out mainly at room temperature. The reaction may be carried out for 6 minutes to 24 hours, preferably for 6 minutes to 16 hours. After completing the reaction, the reaction mixture is concentrated under reduced pressure, washed with water, and subjected to extraction with a suitable organic solvent. The extraction solvent that may be used herein include ether, methylene chloride, chloroform, ethyl acetate, or the like, and most suitably ethyl acetate. After the extraction, the reaction mixture may be subjected to column chromatography to isolate a pure product.

In addition, the imidazole compound of the above Formula 5 used as a starting material for the method according to Reaction Scheme 3 may be obtained by carrying out the method as depicted in the following Reaction Scheme 4.

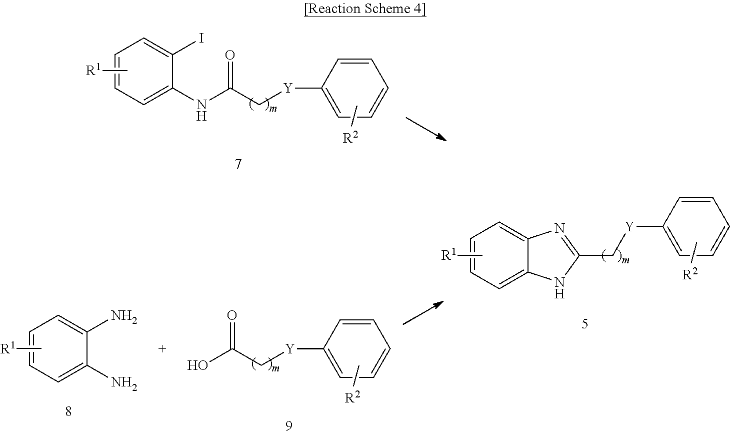

(wherein each of R¹, R², Y, and m is the same as defined in Formula 1).

According to Reaction Scheme 4, the benzimidazole compound represented by Formula 5 may be obtained through two types of cyclization paths.

The first type of cyclization process includes carrying out intramolecular cyclization of the amide compound represented by the above Formula 7 to obtain the benzimidazole compound represented by the above Formula 5. For example, in the intramolecular cyclization, copper iodide may be used as an inorganic catalyst, and L-proline, trans-4-hydroxy-L-proline, 1,10-phenanthroline, etc. may be used as an organic catalyst. In addition, sodium hydroxide, cesium carbonate, potassium carbonate, etc., may be used as a base. Among those, in some embodiments of the present invention, copper iodide is mainly used as an inorganic catalyst, L-proline is used mainly as an organic catalyst, and sodium hydroxide is used mainly as a base. When an intermediate reaction is terminated, the cyclization is completed with an acid catalyst such as acetic acid or sulfuric acid. In some embodiments of the present invention, acetic acid is used mainly. Particular examples of the reaction solvent that may be used include acetonitrile, dimethyl formaldehyde, dimethyl sulfoxide, tetrahydrofuran, or the like. In some embodiments of the present invention, dimethyl sulfoxide is used mainly as a reaction solvent. The reaction temperature is maintained preferably within a range of 60° C. to 100° C., preferably 70° C. to 80° C. The reaction may be carried out for 6-24 hours, preferably for 8-16 hours.

The second type of cyclization process includes subjecting the 1,2-diaminobenzene compound represented by the above Formula 8 and the aromatic carbon acid compound represented by the above Formula 9 to substitution reaction in the presence of an acid catalyst to obtain the benzimidazole compound represented by the above Formula 5. In the substitution reaction, hydrogen chloride or polyphosphoric acid may be used not only as an acid catalyst but also as a reaction solvent. In some embodiments of the present invention, polyphosphoric acid is used mainly. The reaction temperature is maintained preferably within a range of 150° C. to 200° C., preferably 170° C. to 180° C. The reaction may be carried out for 4-8 hours, preferably for 4-6 hours.

In addition, the amide compound of the above Formula 7 used as a starting material for the method according to Reaction Scheme 4 may be obtained by carrying out the method as depicted in the following Reaction Scheme 5.

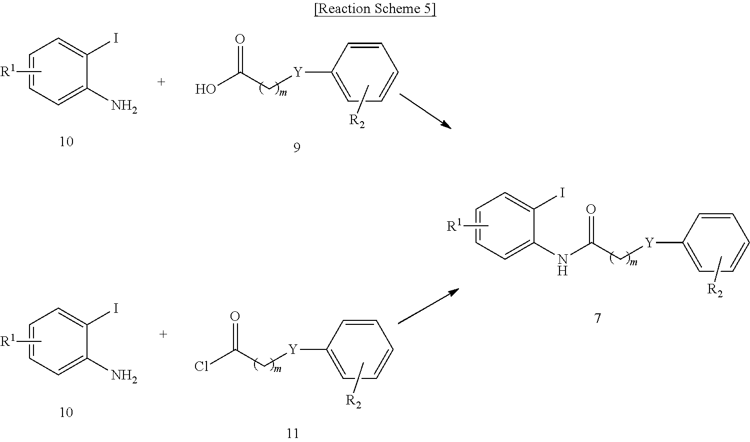

(wherein each of R¹, R², Y, and m is the same as defined in Formula 1).

According to Reaction Scheme 5, the amide compound represented by the above Formula 7 may be obtained through two types of reaction paths.

In the first type of reaction path, the amide compound represented by Formula 7 may be obtained through condensation of the 2-iodoaniline compound represented by the above Formula 10 with the aromatic carbonic acid compound represented by the above Formula 9. The amidating agent that may be used for the condensation may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), (benzotriazol-1-yloxy)tripyrrolidinophosphonium hexafluorophosphate (pyBOP), (benzotriazol-1-yloxy)tris(dimethylamino)phosphonium hexafluorophosphate (BOP), or the like. In some embodiments of the present invention, EDCI is used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) is used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethylene chloride is used mainly as a reaction solvent. The condensation temperature is preferably maintained within a range of 25° C. to 80° C. In some embodiments of the present invention, the condensation is carried out mainly at room temperature. The condensation may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably dimethylene chloride. After the extraction, the reaction mixture may be purified through column chromatography to isolate a pure product.

In the second type of reaction path, the 2-iodoaniline compound represented by the above Formula 10 and the acyl chloride compound having various substituents and represented by the above Formula 11 may be subjected to substitution reaction. Reaction solvents that may be used in the substitution reaction include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, tetrahydrofuran is used mainly. The substitution reaction temperature is maintained mainly within a range of 25° C. to 80° C. In some embodiments of the present invention, the reaction is carried out mainly at room temperature. The reaction may be carried out for 6-24 hours, preferably for 12-16 hours. After completing the reaction, the reaction mixture is concentrated under reduced pressure, washed with an aqueous alkaline solution, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In addition, the benzimidazole compound represented by Formula 1 wherein R³ is —C(O)NR⁴R⁵ may be obtained according to the following Reaction Scheme 6.

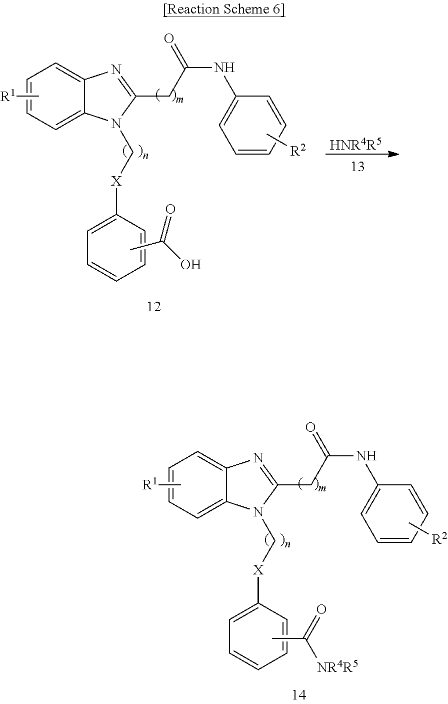

(wherein each of R¹, R², R⁴, R⁵, X, m and n is the same as defined in Formula 1).

According to Reaction Scheme 6, the acid compound represented by the above Formula 12 and the amine compound having various substituents and represented by the above Formula 13 may be subjected to condensation or substitution to obtain the compound represented by the above Formula 14 wherein R³ is —C(O)NR⁴R⁵.

The amidating agent that may be used for the condensation according to Reaction Scheme 6 may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluoro phosphate (HBTU), (benzotriazol-1-yloxy)tripyrrolidino phosphonium hexafluorophosphate (pyBOP), (benzotriazol-1-yloxy)tris(dimethylamino) phosphonium hexafluorophosphate (BOP), or the like. In some embodiments of the present invention, BOP is used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) is used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethyl formamide is used mainly as a reaction solvent. The condensation temperature is preferably maintained within a range of 25° C. to 80° C. In some embodiments of the present invention, the condensation is carried out mainly at room temperature. The condensation may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In a variant, the method of Reaction Scheme 6 may be carried out via substitution reaction. In the substitution reaction, oxalyl chloride may be used as a reagent and dimethyl formamide may be used as a catalyst. Particular examples of the base that may be used include triethylamine (TEA), diisopropylethylamine (DIPEA), 4-methylmorpholine, or the like. In some embodiments of the present invention, DIPEA is used mainly. Reaction solvents that may be used in the substitution reaction include conventional organic solvents, and particular examples thereof include methanol, ethanol, acetonitrile, tetrahydrofuran, 1,2-dichloroethane, methylene chloride, chloroform, dimethylformamide, dimethyl sulfoxide, or the like. In some embodiments of the present invention, methylene chloride and tetrahydrofuran are used mainly. The substitution reaction temperature is maintained preferably within a range of 25° C. to 80° C. In some embodiments of the present invention, the reaction is carried out at room temperature. The reaction may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In addition, the acid compound of the above Formula 12 used as a starting material for the method according to Reaction Scheme 6 may be obtained by carrying out the method as depicted in the following Reaction Scheme 7.

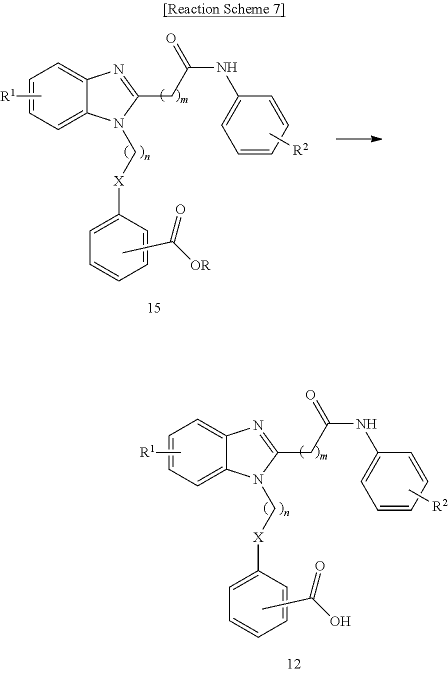

(wherein each of R¹, R², X, m and n is the same as defined in Formula 1, and R is a C₁-C₆ alkyl group).

According to Reaction Scheme 7, the acid compound represented by the above Formula 12 may be obtained by hydrolysis of the ester compound represented by the above Formula 15.

Particular examples of the base that may be used in the hydrolysis according to Reaction Scheme 7 include potassium carbonate, sodium carbonate, lithium hydroxide, sodium hydroxide, potassium hydroxide, or the like. In some embodiments of the present invention, lithium hydroxide and sodium hydroxide are used mainly as a base. As the reaction solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, methanol and tetrahydrofuran are used mainly as a reaction solvent. The hydrolysis temperature is maintained preferably within a range of 25° C. to 80° C. In some embodiments of the present invention, the hydrolysis is carried out mainly at room temperature. The hydrolysis may be carried out for 30 minutes to 24 hours, preferably for 30 minutes to 4 hours. After completing the hydrolysis, the reaction mixture is concentrated under reduced pressure, dissolved in water and acidified with 1N hydrochloric acid. The resultant solid may be obtained by filtering without additional purification process.

In addition, the ester compound of the above Formula 15 used as a starting material for the method according to Reaction Scheme 7 may be obtained by carrying out the method as depicted in the following Reaction Scheme 8.

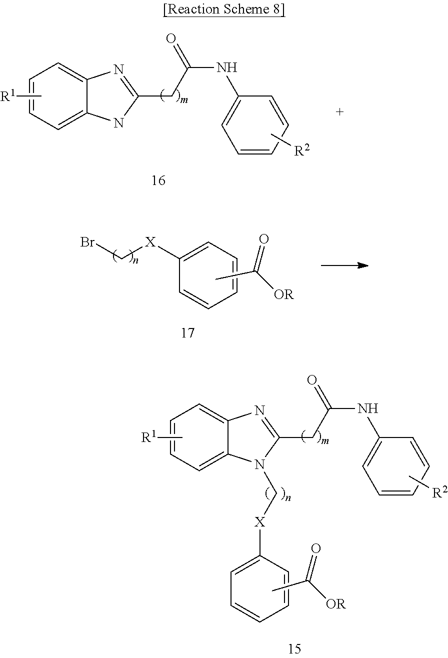

(wherein each of R¹, R², X, m and n is the same as defined in Formula 1, and R is a C₁-C₆ alkyl group).

According to Reaction Scheme 8, the ester compound represented by Formula 15 may be obtained by carrying out a substitution reaction between the imidazole compound represented by the above Formula 16 and the brominated compound represented by the above Formula 17.

Particular examples of the base that may be used in the alkylation according to Reaction Scheme 8 include potassium carbonate, sodium carbonate, lithium hydroxide, sodium hydroxide, potassium hydroxide, lithium hydride, triethylamine (TEA), diisopropylethylamine (DIPEA), 4-methylmorpholine, or the like. In some embodiments of the present invention, diisopropylethylamine (DIPEA) is used mainly as a base. As the reaction solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethyl formamide is used mainly as a reaction solvent. The condensation temperature is maintained preferably within a range of 25° C. to 80° C. In some embodiments of the present invention, the reaction is carried out mainly at 80° C. The reaction may be carried out for 5-10 hours, preferably for 5-6 hours. After completing the reaction, the reaction mixture is concentrated under reduced pressure, washed with water, and subjected to extraction with a suitable organic solvent. The extraction solvent that may be used herein include ether, methylene chloride, chloroform, ethyl acetate, or the like, and most suitably ethyl acetate. After the extraction, the reaction mixture may be subjected to column chromatography to isolate a pure product.

In addition, the imidazole compound of the above Formula 16 used as a starting material for the method according to Reaction Scheme 8 may be obtained by carrying out the method as depicted in the following Reaction Scheme 9.

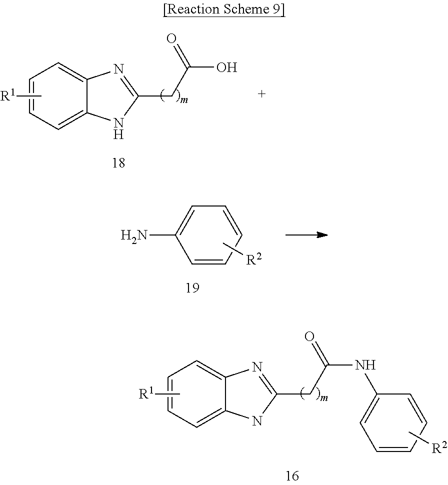

(wherein each of R¹, R², and m is the same as defined in Formula 1).

According to Reaction Scheme 9, the imidazole compound represented by the above Formula 16 may be obtained by condensation between the acid compound represented by the above Formula 18 and the aniline compound represented by the above Formula 19.

The amidating agent that may be used for the condensation according to Reaction Scheme 9 may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluoro phosphate (HBTU), (benzotriazol-1-yloxy)tripyrrolidino phosphonium hexafluorophosphate (pyBOP), (benzotriazol-1-yloxy)tris(dimethylamino) phosphonium hexafluorophosphate (BOP), or the like. In some embodiments of the present invention, HATU is used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) is used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, acetonitrile is used mainly as a reaction solvent. The condensation temperature is maintained preferably within a range of 25° C. to 80° C. In some embodiments of the present invention, the condensation is carried out mainly at room temperature. The condensation may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In addition, the acid compound of the above Formula 18 used as a starting material for the method according to Reaction Scheme 9 may be obtained by carrying out the method as depicted in the following Reaction Scheme 10.

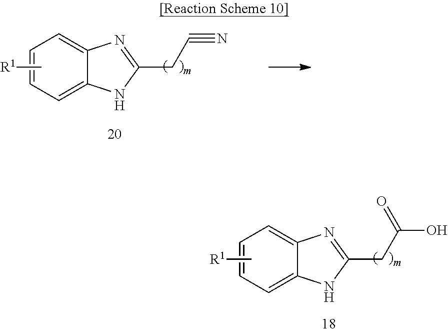

(wherein each of R¹ and m is the same as defined in Formula 1).

According to Reaction Scheme 10, the acid compound represented by the above Formula 18 may be obtained by hydrolysis of the nitrile compound represented by the above Formula 20.

Particular examples of the base that may be used in the hydrolysis according to Reaction Scheme 10 include potassium carbonate, sodium carbonate, lithium hydroxide, sodium hydroxide, potassium hydroxide, or the like. In some embodiments of the present invention, sodium hydroxide is used mainly as a base. As the reaction solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, ethanol is used mainly as a reaction solvent. The hydrolysis temperature is maintained preferably within a range of 25° C. to 80° C. In some embodiments of the present invention, the hydrolysis is carried out mainly at 80° C. The hydrolysis may be carried out for 6-10 hours, preferably for 7-8 hours. After completing the hydrolysis, the reaction mixture is concentrated under reduced pressure, dissolved in water and acidified with 1N hydrochloric acid. The resultant solid may be obtained by filtering without additional purification process.

In addition, the benzimidazole compound represented by Formula 1 wherein Y is —SCH₂— may be obtained according to the following Reaction Scheme 11.

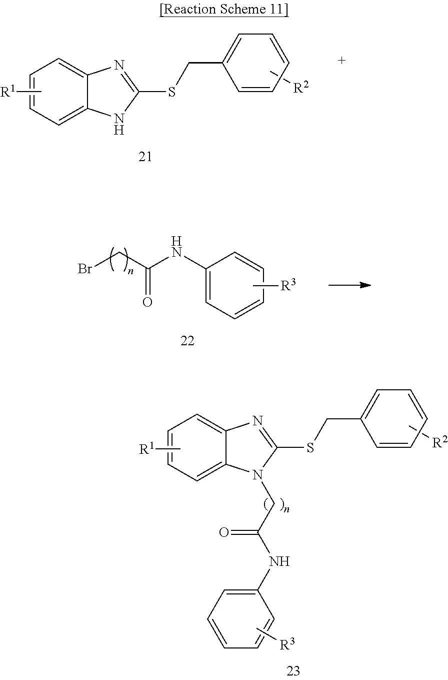

(wherein each of R¹, R², R³, and n is the same as defined in Formula 1).

According to Reaction Scheme 11, the benzimidazole compound represented by the above Formula 23 wherein Y is —SCH₂— may be obtained by carrying out a substitution reaction between the imidazole compound represented by the above Formula 21 and the brominated compound represented by the above Formula 22.

Particular examples of the base that may be used in the substitution according to Reaction Scheme 11 include potassium carbonate, sodium carbonate, lithium hydroxide, sodium hydroxide, potassium hydroxide, triethylamine (TEA), diisopropylethylamine (DIPEA), 4-methylmorpholine (NMM), or the like. In some embodiments of the present invention, potassium carbonate is used mainly as a base. As the reaction solvent, conventional organic solvents may be used, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethyl formamide is used mainly as a reaction solvent. The reaction temperature is maintained preferably within a range of 25° C. to 80° C. In some embodiments of the present invention, the reaction is carried out mainly at room temperature. The reaction may be carried out for 6-24 hours, preferably for 12-16 hours. After completing the reaction, the reaction mixture is concentrated under reduced pressure, washed with water, and subjected to extraction with a suitable organic solvent. The extraction solvent that may be used herein include ether, methylene chloride, chloroform, ethyl acetate, or the like, and most suitably ethyl acetate. After the extraction, the reaction mixture may be subjected to column chromatography to isolate a pure product.

Further, among the imidazole compounds of the above Formula 21 (or the following Formula 25) used as a starting material for the method according to Reaction Scheme 11, the imidazole compound wherein R² is an amide group may be obtained by carrying out the method as depicted in the following Reaction Scheme 12.

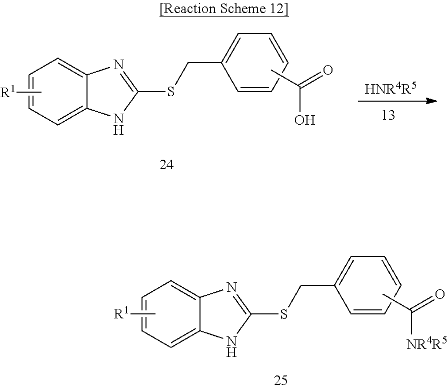

(wherein each of R¹, R⁴, and R⁵ is the same as defined in Formula 1).

According to Reaction Scheme 12, the amide compound represented by the above Formula 25 may be obtained by condensation between the acid compound represented by the above Formula 24 and the amine compound represented by the above Formula 25.

The amidating agent that may be used for the condensation according to Reaction Scheme 12 may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluoro phosphate (HBTU), (benzotriazol-1-yloxy)tripyrrolidino phosphonium hexafluorophosphate (pyBOP), (benzotriazol-1-yloxy)tris(dimethylamino) phosphonium hexafluorophosphate (BOP), or the like. In some embodiments of the present invention, BOP is used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) is used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, dimethyl formamide is used mainly as a reaction solvent. The condensation temperature is maintained preferably within a range of 25° C. to 80° C. In some embodiments of the present invention, the condensation is carried out mainly at room temperature. The condensation may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In addition, the acid compound of the above Formula 24 used as a starting material for the method according to Reaction Scheme 12 may be obtained by carrying out the method as depicted in the following Reaction Scheme 13.

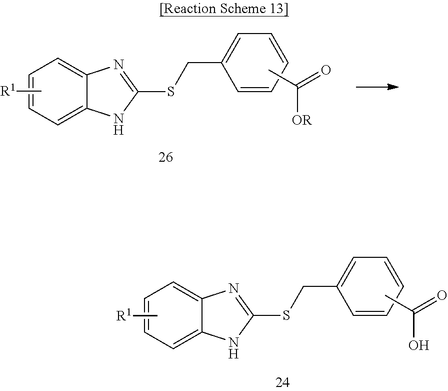

(wherein R¹ is the same as defined in Formula 1, and R is a C₁-C₆ alkyl group).

According to Reaction Scheme 13, the acid compound represented by the above Formula 24 may be obtained by hydrolysis of the ester compound represented by the above Formula 26.

Particular examples of the base that may be used in the hydrolysis according to Reaction Scheme 13 include potassium carbonate, sodium carbonate, lithium hydroxide, sodium hydroxide, potassium hydroxide, or the like. In some embodiments of the present invention, lithium hydroxide is used mainly as a base. As the reaction solvent, conventional organic solvents may be used and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, methanol and tetrahydrofuran are used mainly as a reaction solvent. The hydrolysis temperature is maintained preferably within a range of 25° C. to 80° C. In some embodiments of the present invention, the hydrolysis is carried out mainly at room temperature. The hydrolysis may be carried out for 30 minutes to 24 hours, preferably for 30 minutes to 4 hours. After completing the hydrolysis, the reaction mixture is concentrated under reduced pressure, dissolved in water and acidified with 1N hydrochloric acid. The resultant solid may be obtained by filtering without additional purification process.

In addition, the ester compound of the above Formula 26 used as a starting material for the method according to Reaction Scheme 13 may be obtained by carrying out the method as depicted in the following Reaction Scheme 14.

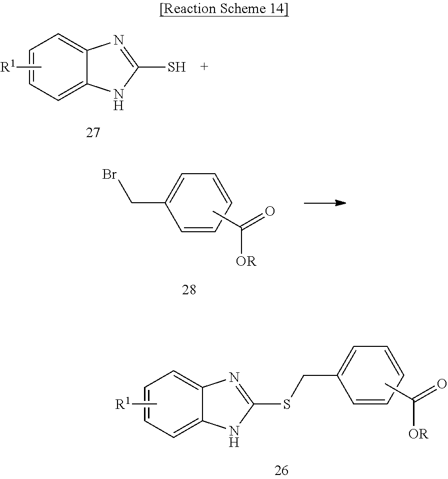

(wherein R¹ is the same as defined in Formula 1, and R is a C₁-C₆ alkyl group).

According to Reaction Scheme 14, the ester compound represented by Formula 26 may be obtained by carrying out reaction between the thiol compound represented by the above Formula 27 and the alkyl bromomethyl benzoate compound represented by the above Formula 28.

Particular examples of the base that may be used in the method according to Reaction Scheme 14 include potassium carbonate, sodium carbonate, lithium hydroxide, sodium hydroxide, potassium hydroxide, sodium hydride, triethylamine (TEA), diisopropylethyl amine (DIPEA), 4-methylmorpholine (NMM) or the like. In some embodiments of the present invention, potassium carbonate is used mainly as a base. As the reaction solvent, water and conventional organic solvents may be used, and particular examples thereof include acetone, methanol, ethanol, acetonitrile, tetrahydrofuran, 1,2-dichloroethane, methylene chloride, dimethylformamide, dimethyl sulfoxide, or the like. In some embodiments of the present invention, acetone is used mainly as a reaction solvent. The reaction temperature is maintained preferably within a range of 25° C. to 80° C. In some embodiments of the present invention, the reaction is carried out mainly at 50° C. The reaction may be carried out for 1-4 hours, preferably for 2-3 hours. After completing the reaction, the reaction mixture is concentrated under reduced pressure, washed with water, and subjected to extraction with a suitable organic solvent. The extraction solvent that may be used herein include ether, methylene chloride, chloroform, ethyl acetate, or the like, and most suitably ethyl acetate. After the extraction, the reaction mixture may be subjected to column chromatography to isolate a pure product.

In addition, the thiol compound of the above Formula 27 used as a starting material for the method according to Reaction Scheme 14 may be obtained by carrying out the method as depicted in the following Reaction Scheme 15.

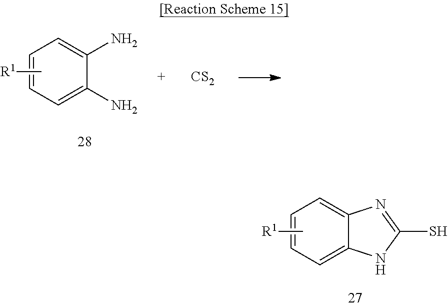

(wherein R¹ is the same as defined above).

According to Reaction Scheme 15, the thiol compound represented by Formula 27 may be obtained through cyclization between the 1,2-diaminobenzene compound represented by the above Formula 28 and carbon disulfide.

As the reaction solvent that may be used in the cyclization according to Reaction Scheme 15, conventional organic solvents may be used, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, ethanol is used mainly as a reaction solvent. The reaction temperature is maintained preferably within a range of 25° C. to 80° C. In some embodiments of the present invention, the reaction is carried out mainly at 60° C. The reaction may be carried out for 5-8 hours, preferably for 5-6 hours. After completing the reaction, the reaction mixture is cooled at room temperature and subjected to filtering without additional purification process.

Further, the amide compound of the above Formula 22 used as a starting material for the method according to Reaction Scheme 11 may be obtained by carrying out the method as depicted in the following Reaction Scheme 16.

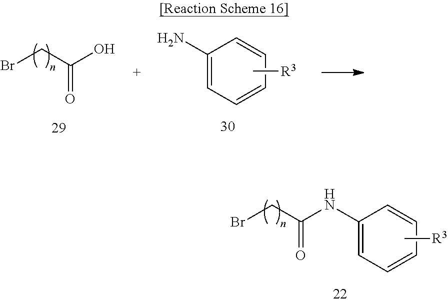

(wherein each of R³ and n is the same as defined above).

According to Reaction Scheme 16, the amide compound represented by the above Formula 22 may be obtained by condensation between the bromoalkanoate compound represented by the above Formula 29 and the aniline compound represented by the above Formula 30.

The amidating agent that may be used for the condensation according to Reaction Scheme 16 may include 1,1′-carbonyldiimidazole (CDI), 3-(ethyliminomethyleneamino)-N,N-dimethylpropane-1-amine (EDCI), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluoro phosphate (HBTU), (benzotriazol-1-yloxy)tripyrrolidinophosphonium hexafluorophosphate (pyBOP), (benzotriazol-1-yloxy)tris(dimethylamino) phosphonium hexafluorophosphate (BOP), or the like. In some embodiments of the present invention, HATU is used mainly as an amidating agent. Particular examples of the base that may be used in the condensation may include triethylamine (TEA), diisopropylethylamine (DIPEA), 4-methylmorpholine (NMM), or the like. In some embodiments of the present invention, triethylamine (TEA) is used mainly as a base. The reaction solvents that may be used in the condensation may include conventional organic solvents, and particular examples thereof include methanol, ethanol, acetonitrile, tetrahydrofuran, 1,2-dichloroethane, methylene chloride, dimethylformamide, dimethyl sulfoxide, or the like. In some embodiments of the present invention, acetonitrile is used mainly as a reaction solvent. The condensation temperature is maintained preferably within a range of 25° C. to 80° C. In some embodiments of the present invention, the condensation is carried out mainly at room temperature. The condensation may be carried out for 6-24 hours, preferably for 12-16 hours. After completing the reaction, the reaction mixture is concentrated under reduced pressure, washed with water, and subjected to extraction with a suitable organic solvent. Particular examples of the extraction solvent include ether, methylene chloride, chloroform and ethyl acetate, and most suitably ethyl acetate. After the extraction, the reaction mixture may be purified through column chromatography to isolate a pure product.

In another aspect, the present invention provides a pharmaceutical composition including the benzimidazole derivative represented by Formula 1 or pharmaceutically acceptable salt thereof as an active ingredient for preventing or treating diseases.

The pharmaceutical composition according to some embodiments of the present invention may be obtained by formulating the benzimidazole derivative represented by Formula 1 or pharmaceutically acceptable salt thereof and other conventional carriers, adjuvants or diluents into a form suitable for oral or parenteral administration by a conventional formulation process. In the case of oral administration, formulations such as tablets, capsules, solutions, syrups or suspensions may be used. In the case of parenteral administration, intraperitoneal, subcutaneous, intramuscular and transdermal injection formulations may be used.

The daily dose of the pharmaceutical composition according to some embodiments of the present invention as a neuro-protective agent effective for mitochondria may be 0.01 to 1000 mg/day, but may depend on ages, weights, sex, administration paths, physical conditions and disease severity of patients. The pharmaceutical compositions may be administered once or several times per day at predetermined time intervals according to the decision made by physicians or pharmacists.

Therefore, the present invention also provides use of the benzimidazole derivative represented by Formula 1 or pharmaceutically acceptable salt thereof, or a pharmaceutical composition including the same for the purpose of prevention or treatment of diseases.

In other words, the benzimidazole derivative represented by Formula 1 or pharmaceutically acceptable salt thereof is an active ingredient as a neuro-protective agent effective for mitochondria, and thus the use includes pharmaceutical use for preventing or treating diseases, including Alzheimer's disease, Parkinson's disease, Huntington's disease, ischemic brain diseases (e.g., stroke), diabetes and schizophrenia.

### EXAMPLES

The examples and experiments will now be described. The following examples and experiments are for illustrative purposes only and not intended to limit the scope of this disclosure.

### Example 1

### N-(2-iodophenyl)benzamide

First, 2-iodoaniline (100 mg, 0.46 mmol) is dissolved into THF (1 mL) under nitrogen and benzoyl chloride (59 μL, 0.51 mmol) is added gradually thereto at 0° C. The reaction mixture is warmed to room temperature and agitated for 24 hours while the reaction progress and result are checked by TLC. After completing the reaction, the reaction mixture is extracted with ethyl acetate and water and 5% aqueous solution of sodium carbonate is added dropwise thereto to perform neutralization, followed by extraction with ethyl acetate. The organic layer is dried with dry magnesium sulfate, followed by filtering. The filtrate is concentrated under reduced pressure to obtain 125 mg of the target compound (yield: 85%).

¹H NMR (300 MHz, CDCl₃-d) δ ppm 8.49 (dd, J=4.74 Hz, 1H) 8.24 (br s, 1H) 7.99 (dd, J=4.13 Hz, 2H) 7.84 (dd, J=4.59 Hz, 1H) 7.57 (m, 3H) 7.43 (t, J=2.22 Hz, 1H) 6.91 (t, J=2.43 Hz, 1H)

### Example 2

### 2-phenyl-1H-benzo[d]imidazole

To a round-bottom flask, N-(2-iodophenyl)benzamide (50 mg, 0.15 mmol), copper iodide (2.86 mg, 0.015 mmol), L-proline (3.45 mg, 0.03 mmol) and sodium hydroxide (9 mg, 0.225 mmol) are introduced and purging is carried out with nitrogen. Then, dimethyl sulfoxide (1 mL) is added thereto to dissolve the reaction mixture and 30% aqueous ammonia (14.3 μL, 0.225 mmol) is added dropwise thereto. The reaction mixture is agitated for 2-3 hours. The reaction progress and result are checked by TLC. Then, acetic acid (1 mL) is added dropwise thereto at room temperature and the reaction mixture is refluxed at 80° C. for 7 hours. The reaction progress and result are checked by TLC. After completing the reaction, the reaction mixture is extracted with ethyl acetate and water and aqueous solution of potassium carbonate is added dropwise thereto to perform neutralization, followed by extraction with ethyl acetate. The organic layer is dried with dry sodium sulfate, followed by filtering. The filtrate is concentrated under reduced pressure and the concentrate is purified by column chromatography (EA:n-hHex=1:2) to obtain 18 mg of the target compound (yield: 60%).

¹H NMR (300 MHz, DMSO-d₆) δ ppm 12.92 (br s, 1H) 8.18 (m, 2H) 7.52 (m, 5H) 7.21 (m, 2H)

### Example 3

### Methyl 2-(2-phenyl-1H-benzo[d]imidazol-1-yl)acetate

2-phenyl-1H-benzo[d]imidazole (50 mg, 0.26 mmol) and sodium hydride (7.5 mg, 0.31 mmol) are dissolved into dimethyl formamide (500 μL) under nitrogen. At room temperature, the reaction mixture is agitated for 1 hour. After adding methyl bromoacetate (28.6 μL, 0.31 mmol) is added thereto, the reaction mixture is agitated for 16 hours at room temperature. The reaction progress and result are checked by TLC. After completing the reaction, water is added to the reaction mixture and is extracted with ethyl acetate. The organic layer is dried with dry magnesium sulfate, followed by filtering. The filtrate is concentrated under reduced pressure and the concentrate is purified by column chromatography (EA:n-Hex=1:2) to obtain 35 mg of the target compound (yield: 50%).

¹H NMR (300 MHz, CDCl₃-d) δ ppm 7.87 (m, 1H) 7.72 (m, 2H) 7.54 (m, 3H) 7.35 (m, 3H) 4.93 (s, 2H) 3.82 (s, 3H)

### Example 4

### 2-(2-phenyl-1H-benzo[d]imidazol-1-yl)acetic acid

Methyl 2-(2-phenyl-1H-benzo[d]imidazol-1-yl)acetate (200 mg, 0.75 mmol) is dissolved into methanol (2 mL) under nitrogen. Then, 1N sodium hydroxide (2.25 mL, 2.25 mmol) is added dropwise thereto gradually at room temperature. The reaction mixture is agitated for 30 minutes while the reaction progress and result are checked by TLC. After completing the reaction, the reaction mixture is concentrated under reduced pressure. Then, 1N hydrochloric acid is added dropwise thereto gradually to acidify the reaction mixture, followed by extraction with ethyl acetate. The organic layer is dried with dry magnesium sulfate, followed by filtering. The filtrate is concentrated under reduced pressure to obtain 150 mg of the target compound (yield: 80%).

¹H NMR (300 MHz, MeOD) δ ppm 7.81 (m, 9H) 5.36 (s, 2H)

### Example 5

### N-(3,5-di-tert-butylphenyl)-2-(2-phenyl-1H-benzo[d]imidazol-1-yl)acetamide

2-(2-phenyl-1H-benzo[d]imidazol-1-yl)acetic acid (20 mg, 0.079 mmol), 3,5-di-tert-butylaniline (21 mg, 0.10 mmol) and O-(7-azabenzotriazol-1-yl)-N,N,N′,N′-tetramethyluronium hexafluorophosphate (HATU, 38 mg, 0.10 mmol) are dissolved into acetonitrile (3 mL) under nitrogen. Triethylamine (TEA, 56 μL, 0.40 mmol) is added dropwise thereto at room temperature, followed by agitation for 16 hours. The reaction progress and result are checked by TLC. After completing the reaction, the reaction mixture is concentrated under reduced pressure. The reaction mixture is extracted with ethyl acetate, and the organic layer is dried with dry magnesium sulfate, followed by filtering. The filtrate is concentrated under reduced pressure and the concentrate is purified by column chromatography (EA:n-Hex=1:1). The resultant product is recrystallized with diethyl ether to obtain 21 mg of the target compound (yield: 60%).

¹H NMR (300 MHz, MeOD) δ ppm 7.89 (m, 3H) 7.58 (m, 4H) 7.44 (m, 2H) 7.34 (m, 2H) 7.24 (m, 1H) 5.08 (s, 2H) 1.30 (s, 18H)

### Example 6

### N-(5-isopropyl-2-methylphenyl)-2-(2-phenyl-1H-benzo[d]imidazol-1-yl)acetamide

Example 5 is repeated to obtain 11 mg of the target compound (yield: 35%), except that 2-(2-phenyl-1H-benzo[d]imidazol-1-yl)acetic acid (20 mg, 0.079 mmol), 5-isopropyl-2-methylaniline (21 mg, 0.10 mmol), HATU (38 mg, 0.10 mmol) and TEA (56 μL, 0.40 mmol) are used.

¹H NMR (300 MHz, MeOD) δ ppm 7.81 (m, 2H) 7.74 (m, 1H) 7.60 (m, 4H) 7.37 (m, 2H) 7.14 (m, 2H) 7.03 (m, 1H) 5.17 (s, 2H) 2.85 (heptet, J=7.01 Hz, 1H) 2.16 (s, 3H) 1.20 (s, 3H) 1.17 (s, 3H)

### Example 7

### N-(3,5-dichlorophenyl)-2-(2-phenyl-1H-benzo[d]imidazol-1-yl)acetamide

Example 5 is repeated to obtain 24 mg of the target compound (yield: 50%), except that 2-(2-phenyl-1H-benzo[d]imidazol-1-yl)acetic acid (30 mg, 0.12 mmol), 3,5-dichloroaniline (25 mg, 0.15 mmol), HATU (91 mg, 0.24 mmol) and TEA (84 μL, 0.60 mmol) are used.

¹H NMR (300 MHz, MeOD) δ ppm 7.75 (m, 3H) 7.58 (m, 5H) 7.50 (m, 1H) 7.36 (m, 2H) 7.18 (m, 1H) 5.11 (s, 2H)

### Example 8

### N-(2-tert-butyl-6-methylphenyl)-2-(2-phenyl-1H-benzo[d]imidazol-1-yl)acetamide

Example 5 is repeated to obtain 6 mg of the target compound (yield: 13%), except that 2-(2-phenyl-1H-benzo[d]imidazol-1-yl)acetic acid (30 mg, 0.12 mmol), 2-methyl-6-tert-butylaniline (78 mg, 0.48 mmol), HATU (274 mg, 0.72 mmol) and TEA (84 μL, 0.48 mmol) are used.

¹H NMR (300 MHz, CDCl₃-d) δ ppm 7.89 (m, 3H) 7.55 (m, 4H) 7.42 (m, 2H) 7.13 (m, 2H) 6.74 (m, 1H) 5.11 (s, 2H) 2.03 (s, 3H) 1.08 (s, 9H)

### Example 9

### 2,5-dichloro-N-(2-iodophenyl)benzamide

Example 1 is repeated to obtain 2.22 g of the target compound (yield: 85%), except that 2-iodoaniline (1.5 g, 6.85 mmol) and dichlorobenzoyl chloride (1.58 g, 7.53 mmol) are used.

¹H NMR (300 MHz, CDCl₃-d) δ ppm 8.38 (d, J=4.03 Hz, 1H) 8.16 (br s, 1H) 7.85 (dd, J=4.59 Hz, 1H) 7.79 (s, 1H) 7.43 (m, 3H) 6.94 (m, 1H)

### Example 10

### 2-(2,5-dichlorophenyl)-1H-benzo[d]imidazole

Example 2 is repeated to obtain 810 mg of the target compound (yield: 55%), except that 2,5-dichloro-N-(2-iodophenyl)benzamide (2.2 g, 5.61 mmol), cooper iodide (100 mg, 0.56 mmol), L-proline (130 mg, 1.12 mmol), sodium hydroxide (340 mg, 8.42 mmol), 30% aqueous ammonia (1.1 mL, 8.42 mmol) and acetic acid are used.

¹H NMR (300 MHz, CDCl₃-d) δ ppm 10.29 (br s, 1H) 8.50 (m, 1H) 7.87 (br s, 1H) 7.56 (br s, 1H) 7.39 (m, 4H)

### Example 11

### Methyl 2-(2-(2,5-dichlorophenyl-1H-benzo[d]imidazol-1-yl)acetate

Example 3 is repeated to obtain 400 mg of the target compound (yield: 39%), except that 2-(2,5-dichlorophenyl)-1H-benzo[d]imidazole (800 mg, 3.04 mmol), sodium hydride (118 mg, 3.65 mmol) and methyl bromoacetate (540 μL, 3.65 mmol) are used.

¹H NMR (300 MHz, CDCl₃-d) δ ppm 7.88 (m, 1H) 7.62 (s, 1H) 7.44 (m, 2H) 7.37 (m, 3H) 4.80 (s, 2H) 3.72 (s, 3H)

### Example 12

### 2-(2-(2,5-dichlorophenyl-1H-benzo[d]imidazol-1-yl)acetic acid

Example 4 is repeated to obtain 310 mg of the target compound (yield: 80%), except that methyl 2-(2-(2,5-dichlorophenyl)-1H-benzo[d]imidazol-1-yl)acetate (400 mg, 1.19 mmol) and 1N sodium hydroxide (3.58 mL, 3.58 mmol) are used.

¹H NMR (300 MHz, DMSO-d₆) δ ppm 13.31 (br s, 1H) 7.72 (m, 3H) 7.63 (m, 1H) 7.58 (m, 1H) 7.33 (m, 2H) 4.91 (s, 2H)

### Example 13

### N-(3,5-di-tert-butylphenyl)-2-(2-(2,5-dichlorophenyl)-1H-benzo[d]imidazol-1-yl)acetamide

Example 5 is repeated to obtain 34 mg of the target compound (yield: 72%), except that 2-(2-(2,5-dichlorophenyl)-1H-benzo[d]imidazol-1-yl)acetic acid (30 mg, 0.09 mmol), 3,5-di-tert-butylaniline (25 mg, 0.12 mmol), HATU (46 mg, 0.12 mmol) and TEA (63 μL, 0.45 mmol) are used.

¹H NMR (300 MHz, CDCl₃-d) δ ppm 7.91 (m, 1H) 7.63 (m, 1H) 7.46 (m, 5H) 7.20 (m, 1H) 7.16 (m, 3H) 4.84 (s, 2H) 1.28 (s, 18H)

### Example 14

### N-(5-isopropyl-2-methylphenyl)-2-(2-phenyl-1H-benzo[d]imidazol-1-yl)acetamide

Example 5 is repeated to obtain 23 mg of the target compound (yield: 33%), except that 2-(2-(2,5-dichlorophenyl)-1H-benzo[d]imidazol-1-yl)acetic acid (50 mg, 0.17 mmol), 5-isopropyl-2-methylaniline (35 μg, 0.22 mmol), HATU (84 mg, 0.22 mmol) and TEA (119 μL, 0.85 mmol) are used.

¹H NMR (300 MHz, MeOD) δ ppm 7.75 (m, 1H) 7.66 (m, 4H) 7.42 (m, 2H) 7.10 (m, 2H) 7.04 (m, 1H) 5.08 (s, 2H) 2.83 (heptet, J=7.01 Hz, 1H) 2.09 (s, 3H) 1.18 (s, 3H) 1.13 (s, 3H)

### Example 15

### N-(3,5-dichlorophenyl)-2-(2-(2,5-dichlorophenyl)-1H-benzo[d]imidazol-1-yl)acetamide

Example 5 is repeated to obtain 3 mg of the target compound (yield: 4%), except that 2-(2-(2,5-dichlorophenyl)-1H-benzo[d]imidazol-1-yl)acetic acid (45 mg, 0.14 mmol), 3,5-dichloroaniline (29 mg, 0.18 mmol), HATU (160 mg, 0.42 mmol) and TEA (98 μL, 0.7 mmol) are used.

¹H NMR (300 MHz, MeOD) δ ppm 7.76 (m, 1H) 7.67 (m, 1H) 7.63 (m, 3H) 7.58 (m, 2H) 7.51 (m, 2H) 7.42 (m, 2H) 7.16 (m, 1H) 5.01 (s, 2H)

### Example 16

### N-(2-tert-butyl-6-methylphenyl)-2-(2-(2,5-dichlorophenyl)-1H-benzo[d]imidazol-1-yl)acetamide

Example 5 is repeated to obtain 7 mg of the target compound (yield: 19%), except that 2-(2-(2,5-dichlorophenyl)-1H-benzo[d]imidazol-1-yl)acetic acid (26 mg, 0.08 mmol), 2-methyl-6-tert-butylaniline (16 mg, 0.1 mmol), HATU (38 mg, 0.1 mmol) and TEA (56 μL, 0.4 mmol) are used.

¹H NMR (300 MHz, CDCl₃-d) δ ppm 7.92 (d, J=3.93 Hz, 1H) 7.71 (s, 1H) 7.53 (m, 3H) 7.45 (m, 2H) 7.15 (m, 3H) 6.67 (s, 1H) 5.00 (s, 2H) 2.02 (s, 3H) 1.08 (s, 9H)

### Example 17

### N-(2-iodophenyl)-2,5-dimethoxybenzamide

2-iodoaniline (286 mg, 1.31 mmol), 1-ethyl-3-(3-dimethylaminopropyl)-carbodiimide hydrochloride (EDCI, 418 mg, 2.1 mmol), 1-hydroxybenzotriazole (HOBT, 250 mg, 1.85 mmol) and 2,5-dimethoxybenzoic acid (200 mg, 1.09 mmol) are dissolved into methylene chloride (8 mL) under nitrogen, 4-methylmorpholine (NMM, 400 μL, 1.85 mmol) is added thereto, and the reaction mixture is agitated for 16 hours at room temperature. The reaction progress and result are checked by TLC. After completing the reaction, the reaction mixture is concentrated under reduced pressure. After extracting the reaction mixture with ethyl acetate and water, the organic layer is dried with dry magnesium sulfate, followed by filtering. The filtrate is concentrated under reduced pressure and the concentrate is purified by column chromatography (EA:n-hHex=1:2) to obtain 75 mg of the target compound (yield: 30%).

¹H NMR (300 MHz, CDCl₃-d) δ ppm 10.31 (br s, 1H) 8.50 (d, J=4.86 Hz, 1H) 7.85 (m, 2H) 7.37 (m, 1H) 7.08 (m, 1H) 7.01 (m, 1H) 6.86 (m, 1H) 4.08 (s, 3H) 3.86 (s, 3H)

### Example 18

### 2-(2,5-dimethoxyphenyl)-1H-benzo[d]imidazole

Example 2 is repeated to obtain 336 mg of the target compound (yield: 77%), except that N-(2-iodophenyl)-2,5-dimethoxybenzamide (660 mg, 1.72 mmol), cooper iodide (32 mg, 0.17 mmol), L-proline (39 mg, 0.34 mmol), sodium hydroxide (100 mg, 2.58 mmol), 30% aqueous ammonia (0.3 mL, 2.58 mmol) and acetic acid (27 mL) are used.

¹H NMR (300 MHz, CDCl₃-d) δ ppm 8.11 (m, 1H) 7.66 (m, 2H) 7.27 (m, 2H) 6.98 (m, 2H) 4.01 (s, 3H) 3.82 (s, 3H)

### Example 19

### Methyl 2-(2-(2,5-dimethoxyphenyl-1H-benzo[d]imidazol-1-yl)acetate

Example 3 is repeated to obtain 97 mg of the target compound (yield: 50%), except that 2-(2,5-dimethoxyphenyl)-1H-benzo[d]imidazole (150 mg, 0.59 mmol), sodium hydride (17 mg, 0.71 mmol) and methyl bromoacetate (62 μL, 0.71 mmol) are used.

¹H NMR (300 MHz, CDCl₃-d) δ ppm 7.84 (m, 1H) 7.31 (m, 3H) 7.21 (m, 1H) 7.04 (m, 1H) 6.95 (m, 1H) 4.77 (s, 2H) 3.81 (s, 3H) 3.73 (s, 3H) 3.69 (s, 3H)

### Example 20

### 2-(2-(2,5-dimetoxyphenyl-1H-benzo[d]imidazol-1-yl)acetic acid

Example 4 is repeated to obtain 70 mg of the target compound (yield: 95%), except that methyl 2-(2-(2,5-dimethoxyphenyl)-1H-benzo[d]imidazol-1-yl)acetate (100 mg, 0.3 mmol) and 1N sodium hydroxide (0.1 mL, 0.1 mmol) are used.

¹H NMR (300 MHz, MeOD) δ ppm 7.86 (m, 2H) 7.66 (m, 2H) 7.29 (m, 3H) 5.12 (s, 2H) 3.85 (s, 6H)

### Example 21

### N-(5-(biphenyl-4-yloxy)-2-fluorophenyl)-2-(2-(2,5-dimethoxyphenyl)-1H-benzo[d]imidazol-1-yl)acetamide

Example 5 is repeated to obtain 1 mg of the target compound (yield: 3%), except that 2-(2-(2,5-dimethoxyphenyl)-1H-benzo[d]imidazol-1-yl)acetic acid (16 mg, 0.05 mmol), 5-(biphenyl-4-yloxy)-2-fluoroaniline (20 mg, 0.07 mmol), HATU (23 mg, 0.06 mmol) and TEA (28 μL, 0.20 mmol) are used.

¹H NMR (300 MHz, CDCl₃-d) δ ppm 8.40 (dd, J=5.18, 1H) 8.14 (s, 1H) 7.70 (m, 1H) 7.47 (m, 4H) 7.29 (m, 2H) 7.17 (m, 6H) 7.04 (m, 2H) 6.91 (m, 1H) 6.45 (d, J=4.36, 2H) 4.74 (s, 2H) 3.79 (s, 3H) 3.48 (s, 3H)

### Example 22

### 2-(2-(2,5-dimethoxyphenyl)-1H-benzo[d]imidazol-1-yl)-N-(5-isopropyl-2-methylphenyl)acetamide

Example 5 is repeated to obtain 6 mg of the target compound (yield: 11%), except that 2-(2-(2,5-dimethoxyphenyl)-1H-benzo[d]imidazol-1-yl)acetic acid (50 mg, 0.13 mmol), 5-isopropyl-2-methylaniline (30 μg, 0.19 mmol), HATU (65 mg, 0.17 mmol) and TEA (73 μL, 0.52 mmol) are used.

¹H NMR (300 MHz, MeOD) δ ppm 7.72 (d, J=4.18 Hz, 1H) 7.60 (d, J=3.68, 1H) 7.35 (q, J=7.28, 2H) 7.14 (m, 5H) 7.00 (m, 1H) 5.05 (s, 2H) 3.80 (s, 6H) 2.83 (heptet, J=7.01 Hz, 1H) 2.05 (s, 3H) 1.21 (s, 3H) 1.06 (s, 3H)

### Example 23

### N-(3,5-dichlorophenyl)-2-(2-(2,5-dimethoxyphenyl)-1H-benzo[d]imidazol-1-yl)acetamide

Example 5 is repeated to obtain 10 mg of the target compound (yield: 17%), except that 2-(2-(2,5-dimethoxyphenyl)-1H-benzo[d]imidazol-1-yl)acetic acid (45 mg, 0.14 mmol), 3,5-dichloroaniline (29 mg, 0.18 mmol), HATU (160 mg, 0.42 mmol) and TEA (98 μL, 0.7 mmol) are used.

¹H NMR (300 MHz, MeOD) δ ppm 7.55 (m, 1H) 7.36 (m, 2H) 7.27 (m, 2H) 7.15 (m, 4H) 4.95 (s, 2H) 3.79 (s, 3H) 3.69 (s, 3H)

### Example 24

### N-(2-iodophenyl)-2-phenylacetamide

Example 1 is repeated to obtain 4 g of the target compound (yield: 87%), except that 2-iodoaniline (3 g, 13.7 mmol) and phenylacetyl chloride (1.99 mL, 15.07 mmol) are used.

¹H NMR (300 MHz, CDCl₃-d) δ ppm 8.28 (d, J=3.71 Hz, 1H) 7.69 (dd, J=4.68 Hz, 1H) 7.39 (m, 7H) 6.82 (dt, J=3.75 Hz, 1H) 3.81 (s, 2H)

### Example 25

### 2-benzyl-1H-benzo[d]imidazole

Example 2 is repeated to obtain 560 mg of the target compound (yield: 45%), except that N-(2-iodophenyl)-2-phenylacetamide (1 g, 2.96 mmol), copper iodide (60 mg, 0.3 mmol), L-proline (70 mg, 0.6 mmol), sodium hydroxide (179 mg, 4.44 mmol), 30% aqueous ammonia (580 μL, 4.44 mmol) and acetic acid (10 mL) are used.

¹H NMR (300 MHz, CDCl₃-d) δ ppm 7.54 (m, 2H) 7.34 (m, 5H) 7.24 (m, 2H) 4.34 (s, 2H)

### Example 26

### Methyl 2-(2-benzyl-1H-benzo[d]imidazol-1-yl)acetate

Example 3 is repeated to obtain 35 mg of the target compound (yield: 50%), except that 2-benzyl-1H-benzo[d]imidazole (50 mg, 0.24 mmol), sodium hydride (7 mg, 0.28 mmol) and methyl bromoacetate (28.8 μL, 0.31 mmol) are used.

¹H NMR (300 MHz, CDCl₃-d) δ ppm 7.81 (m, 1H) 7.25 (m, 8H) 4.71 (s, 2H) 4.34 (s, 2H) 3.60 (s, 3H)

### Example 27

### 2-(2-benzyl-1H-benzo[d]imidazol-1-yl)acetic acid

Example 4 is repeated to obtain 100 mg of the target compound (yield: 70%), except that methyl 2-(2-benzyl-1H-benzo[d]imidazol-1-yl)acetate (200 mg, 0.75 mmol) and 1N sodium hydroxide (2.25 mL, 2.25 mmol) are used.

¹H NMR (300 MHz, MeOD) δ ppm 7.63 (m, 2H) 7.43 (m, 2H) 7.34 (m, 4H) 5.00 (s, 2H) 4.45 (s, 2H)

### Example 28

### 2-(2-benzyl-1H-benzo[d]imidazol-1-yl)-N-(3,5-di-tert-butylphenyl)acetamide

Example 5 is repeated to obtain 37 mg of the target compound (yield: 72%), except that 2-(2-benzyl-1H-benzo[d]imidazol-1-yl)acetic acid (30 mg, 0.11 mmol), 3,5-di-tert-butylaniline (30 mg, 0.15 mmol), HATU (57 mg, 0.15 mmol) and TEA (77 μL, 0.55 mmol) are used.

¹H NMR (300 MHz, CDCl₃-d) δ ppm 7.84 (m, 1H) 7.31 (m, 7H) 7.19 (m, 2H) 7.05 (s, 2H) 7.00 (s, 1H) 4.80 (s, 2H) 4.32 (s, 2H) 1.27 (s, 18H)

### Example 29

### 2-(2-benzyl-1H-benzo[d]imidazol-1-yl)-N-(5-isopropyl-2-methylphenyl)acetamide

Example 5 is repeated to obtain 27 mg of the target compound (yield: 73%), except that 2-(2-(benzyl-1H-benzo[d]imidazol-1-yl)acetic acid (25 mg, 0.09 mmol), 5-isopropyl-2-methylaniline (21 mg, 0.12 mmol), HATU (46 mg, 0.12 mmol) and TEA (63 μL, 0.45 mmol) are used.

¹H NMR (300 MHz, CDCl₃-d) δ ppm 7.85 (m, 1H) 7.27 (m, 7H) 6.92 (m, 2H) 6.56 (s, 1H) 4.86 (s, 2H) 4.38 (s, 2H) 2.83 (heptet, J=7.01 Hz, 1H) 1.58 (s, 3H) 1.22 (s, 3H) 1.20 (s, 3H)

### Example 30

### 2-(2-benzyl-1H-benzo[d]imidazol-1-yl)-N-(3,5-dichlorophenyl)acetamide

Example 5 is repeated to obtain 3.5 g of the target compound (yield: 9%), except that 2-(2-(benzyl-1H-benzo[d]imidazol-1-yl)acetic acid (25 mg, 0.09 mmol), 3,5-dichloroaniline (19 mg, 0.12 mmol), HATU (46 mg, 0.12 mmol) and TEA (63 μL, 0.45 mmol) are used.

¹H NMR (300 MHz, CDCl₃-d) δ ppm 7.85 (m, 1H) 7.34 (m, 7H) 7.18 (m, 1H) 7.06 (m, 1H) 7.02 (m, 2H) 6.62 (s, 1H) 4.84 (s, 2H) 4.31 (s, 2H)

### Example 31

### 2-(2-benzyl-1H-benzo[d]imidazol-1-yl)-N-(5-(biphenyl-4-yloxy)-2-fluorophenyl)acetamide

Example 5 is repeated to obtain 26 mg of the target compound (yield: 44%), except that 2-(2-(benzyl-1H-benzo[d]imidazol-1-yl)acetic acid (30 mg, 0.11 mmol), 5-(biphenyl-4-yloxy)-2-fluoroaniline (42 mg, 0.15 mmol), HATU (57 mg, 0.15 mmol) and TEA (77 μL, 0.55 mmol) are used.

¹H NMR (300 MHz, CDCl₃-d) δ ppm 8.05 (dd, J=5.13, 1H) 7.75 (d, J=4.00, 1H) 7.56 (d, J=3.64, 2H) 7.47 (t, J=9.00, 2H) 7.39 (m, 3H) 7.36 (s, 1H) 7.18 (m, 5H) 7.09 (m, 3H) 6.83 (m, 2H) 6.47 (d, J=4.50, 2H) 4.76 (s, 2H) 4.26 (s, 2H)

### Example 32

### 2-(2-benzyl-1H-benzo[d]imidazol-1-yl)-N-(2-(tert-butyl-6-methylphenyl)acetamide

Example 5 is repeated to obtain 7 mg of the target compound (yield: 22%), except that 2-(2-(benzyl-1H-benzo[d]imidazol-1-yl)acetic acid (20 mg, 0.075 mmol), 2-methyl-6-tert-butylaniline (49 mg, 0.3 mmol), HATU (114 mg, 0.30 mmol) and TEA (114 μL, 0.15 mmol) are used.

¹H NMR (300 MHz, CDCl₃-d) δ ppm 7.84 (m, 1H) 7.34 (m, 8H) 7.17 (m, 4H) 6.61 (s, 1H) 4.87 (s, 2H) 4.46 (s, 2H) 2.17 (s, 3H) 1.14 (s, 9H)

### Example 33

### 2-(3,4-dichlorophenyl)-N-(2-iodophenyl)acetamide

Example 17 is repeated to obtain 5 g of the target compound (yield: 63%), except that 2-iodoaniline (5.13 g, 23.4 mmol), EDCI (7.48 g, 39 mmol), HOBT (4.46 g, 33 mmol), 2-(3,4-dichlorophenyl)acetic acid (4.00 g, 19.5 mmol), NMM (3.6 mL, 33 mmol) are used.

¹H NMR (300 MHz, DMSO-d₆) δ ppm 9.68 (s, 1H) 7.88 (d, J=3.85, 1H) 7.65 (s, 1H) 7.60 (d, J=4.12, 1H) 7.39 (m, 3H) 6.99 (m, 1H) 3.73 (s, 2H)

### Example 34

### 2-(3,4-dichlorobenzyl)-1H-benzo[d]imidazole

Example 2 is repeated to obtain 400 mg of the target compound (yield: 30%), except that 2-(3,4-dichlorophenyl)-N-(2-iodophenyl)acetamide (2.00 g, 4.93 mmol), copper iodide (93 mg, 0.49 mmol), L-proline (114 mg, 0.99 mmol), sodium hydroxide (300 mg, 7.40 mmol), 30% aqueous ammonia (470 μL, 7.40 mmol) and acetic acid (20 mL) are used.

¹H NMR (400 MHz, ACETONE-d₆) δ ppm 11.98 (s, 1H) 8.16 (m, 1H) 8.01 (m, 1H) 7.52 (m, 3H) 7.25 (m, 2H) 4.73 (s, 2H)

### Example 35

### Methyl 2-(2-(3,4-dichlorobenzyl)-1H-benzo[d]imidazol-1-yl)acetate

Example 3 is repeated to obtain 47 mg of the target compound (yield: 21%), except that 2-(3,4-dichlorobenzyl)-1H-benzo[d]imidazole (180 mg, 0.65 mmol), sodium hydride (19 mg, 0.78 mmol) and methyl bromoacetate (78 μL, 0.84 mmol) are used.

¹H NMR (300 MHz, CDCl₃-d) δ ppm 7.89 (m, 1H) 7.37 (s, 1H) 7.34 (m, 1H) 7.29 (m, 2H) 7.22 (m, 1H) 6.96 (m, 1H) 4.71 (s, 2H) 4.25 (s, 2H) 3.64 (s, 3H)

### Example 36

### 2-(2-(3,4-dichlorobenzyl)-1H-benzo[d]imidazol-1-yl)acetic acid

Example 4 is repeated to obtain 100 mg of the target compound (yield: 70%), except that methyl 2-(2-(3,4-dichlorobenzyl)-1H-benzo[d]imidazol-1-yl)acetate (200 mg, 0.75 mmol) and 1N sodium hydroxide (2.25 mL, 2.25 mmol) are used.

¹H NMR (300 MHz, MeOD) δ ppm 7.67 (m, 1H) 7.54 (m, 2H) 7.50 (s, 1H) 7.38 (m, 2H) 7.28 (m, 1H) 5.05 (s, 2H) 4.41 (s, 2H)

### Example 37

### N-(3,5-di-tert-butylphenyl)-2-(2-(3,4-dichlorobenzyl)-1H-benzo[d]imidazol-1-yl)acetamide

Example 5 is repeated to obtain 26 mg of the target compound (yield: 83%), except that 2-(2-(3,4-dichlorobenzyl)-1H-benzo[d]imidazol-1-yl)acetic acid (20 mg, 0.06 mmol), 3,5-di-tert-butylaniline (18 mg, 0.09 mmol), HATU (46 mg, 0.12 mmol) and TEA (25 μL, 0.18 mmol) are used.

¹H NMR (300 MHz, CDCl₃-d) δ ppm 7.88 (m, 1H) 7.43 (s, 1H) 7.37 (m, 4H) 7.67 (m, 2H) 7.10 (s, 2H) 6.81 (brs, 1H) 4.83 (s, 2H) 4.32 (s, 2H) 1.29 (s, 18H)

### Example 38

### 2-(2-(3,4-dichlorobenzyl)-1H-benzo[d]imidazol-1-yl)-N-(5-isopropyl-2-methylphenyl)acetamide

Example 5 is repeated to obtain 6 mg of the target compound (yield: 20%), except that 2-(2-(3,4-dichlorobenzyl)-1H-benzo[d]imidazol-1-yl)acetic acid (20 mg, 0.06 mmol), 5-isopropyl-2-methylaniline (14 mg, 0.09 mmol), HATU (46 mg, 0.12 mmol) and TEA (17 μL, 0.12 mmol) are used.

¹H NMR (300 MHz, CDCl₃-d) δ ppm 8.02 (m, 1H) 7.66 (m, 3H) 7.33 (m, 2H) 7.22 (m, 1H) 7.13 (m, 2H) 7.02 (s, 1H) 5.17 (s, 2H) 4.40 (s, 2H) 2.83 (heptet, J=7.01 Hz, 1H) 2.16 (s, 3H) 1.22 (s, 3H) 1.20 (s, 3H)

### Example 39

### 2-(2-(3,4-dichlorobenzyl)-1H-benzo[d]imidazol-1-yl)-N-(3,5-dichlorophenyl)acetate

Example 5 is repeated to obtain 4 mg of the target compound (yield: 5%), except that 2-(2-(3,4-dichlorobenzyl)-1H-benzo[d]imidazol-1-yl)acetic acid (27 mg, 0.08 mmol), 3,5-dichloroaniline (21 mg, 0.12 mmol), HATU (61 mg, 0.16 mmol) and TEA (23 μL, 0.16 mmol) are used.

¹H NMR (300 MHz, CDCl₃-d) δ ppm 8.02 (m, 1H) 7.66 (m, 3H) 7.33 (m, 2H) 7.22 (m, 1H) 7.13 (m, 2H) 7.02 (s, 1H) 5.17 (s, 2H) 4.40 (s, 2H) 2.83 (heptet, J=7.01 Hz, 1H) 2.16 (s, 3H) 1.22 (s, 3H) 1.20 (s, 3H)

### Example 40

### 2-(2,6-dichlorobenzyl)-1H-benzo[d]imidazole

Benzene-1,2-diamine (500 mg, 3.5 mmol), 2-(2,6-dichlorophenyl)acetic acid (863 mg, 4.21 mmol) and polyphosphoric acid (PPA, 2.6 g, 12.63 mmol) are introduced to a round-bottom flask and the reaction mixture is agitated for 4 hours at 175° C. under nitrogen. After cooling the reaction mixture to room temperature, it is neutralized and washed with 7% NH₄OH. After filtering the suspended materials, 1 g of the target compound is obtained (yield: 92%).

¹H NMR (300 MHz, CDCl₃-d) δ ppm 7.42 (d, J=4.00, 2H) 7.22 (m, 2H) 7.06 (m, 1H) 6.96 (brs, 1H) 6.79 (m, 2H) 4.17 (s, 2H)

### Example 41

### Methyl 2-(2-(2,6-dichlorobenzyl)-1H-benzo[d]imidazol-1-yl)acetate

Example 3 is repeated to obtain 43 mg of the target compound (yield: 90%), except that 2-(2,6-dichlorobenzyl)-1H-benzo[d]imidazole (50 mg, 0.24 mmol), sodium hydride (7 mg, 0.28 mmol) and methyl bromoacetate (28.8 μL, 0.31 mmol) are used.

¹H NMR (300 MHz, CDCl₃-d) δ ppm 7.75 (m, 1H) 7.39 (d, J=3.97, 2H) 7.24 (m, 4H) 4.97 (s, 2H) 4.54 (s, 2H) 3.76 (s, 3H)

### Example 42

### 2-(2-(2,6-dichlorobenzyl)-1H-benzo[d]imidazol-1-yl)acetic acid

Example 4 is repeated to obtain 180 mg of the target compound (yield: 90%), except that methyl 2-(2-(2,6-dichlorobenzyl)-1H-benzo[d]imidazol-1-yl)acetate (200 mg, 0.75 mmol) and 1N sodium hydride (2.25 mL, 2.25 mmol) are used.

¹H NMR (300 MHz, DMSO-d₆) δ ppm 13.25 (brs, 1H) 7.33 (m, 4H) 7.38 (t, J=6.00, 1H) 7.16 (m, 2H) 5.25 (s, 2H) 4.46 (s, 2H)

### Example 43

### N-(3,5-di-tert-butylphenyl)-2-(2-(2,6-dichlorobenzyl)-1H-benzo[d]imidazol-1-yl)acetamide

Example 5 is repeated to obtain 6 mg of the target compound (yield: 19%), except that 2-(2-(2,6-dichlorobenzyl)-1H-benzo[d]imidazol-1-yl)acetic acid (20 mg, 0.06 mmol), 3,5-di-tert-butylaniline (18 mg, 0.10 mmol), HATU (46 mg, 0.12 mmol) and TEA (17 μL, 0.12 mmol) are used.

¹H NMR (300 MHz, CDCl₃-d) δ ppm 7.82 (m, 1H) 7.34 (m, 5H) 7.18 (m, 4H) 7.00 (brs, 1H) 5.00 (s, 2H) 4.62 (s, 2H) 1.29 (s, 18H)

### Example 44

### 2-(2-(2,6-dichlorobenzyl)-1H-benzo[d]imidazol-1-yl)-N-(5-isopropyl-2-methylphenyl)acetamide

Example 5 is repeated to obtain 33 mg of the target compound (yield: 95%), except that 2-(2-(2,6-dichlorobenzyl)-1H-benzo[d]imidazol-1-yl)acetic acid (20 mg, 0.079 mmol), 5-isopropyl-2-methylaniline (21 mg, 0.10 mmol), HATU (38 mg, 0.10 mmol) and TEA (56 μL, 0.40 mmol) are used.

¹H NMR (300 MHz, DMSO-d₆) δ ppm 9.85 (brs, 1H) 7.52 (m, 4H) 7.38 (m, 2H) 7.17 (m, 3H) 7.00 (m, 1H) 5.32 (s, 2H) 4.55 (s, 2H) 2.82 (heptet, J=7.01 Hz, 1H) 2.22 (s, 3H) 1.16 (s, 3H) 1.15 (s, 3H)

### Example 45

### 2-(2,5-dimethoxyphenyl)-N-(2-iodophenyl)-2-phenyl acetamide

Example 1 is repeated to obtain 3.0 g of the target compound (yield: 83%), except that 2-iodoaniline (2 g, 9.10 mmol) and (2,5-dimethoxy phenyl)acetyl chloride (1.75 mL, 10.01 mmol) are used.

¹H NMR (300 MHz, CDCl₃-d) δ ppm 8.25 (dd, J=4.20 Hz, 1H) 7.41 (brs, 1H) 7.71 (dd, J=4.05 Hz, 1H) 7.32 (m, 1H) 6.92 (d, J=0.20 Hz, 1H) 6.84 (m, 3H) 3.89 (s, 3H) 3.80 (s, 3H) 3.76 (s, 2H)

### Example 46

### 2-(2,5-dimethoxybenzyl)-1H-benzo[d]imidazole

Example 2 is repeated to obtain 200 mg of the target compound (yield: 30%), except that 2-(2,5-dimethoxyphenyL)-N-(2-iodophenyl)acetamide (1 g, 2.51 mmol), copper iodide (48 mg, 0.25 mmol), L-proline (48 mg, 0.50 mmol), sodium hydroxide (151 mg, 3.77 mmol), 30% aqueous ammonia (500 μL, 3.77 mmol) and acetic acid (10 mL) are used.

¹H NMR (300 MHz, CDCl₃-d) δ ppm 7.52 (m, 1H) 7.20 (m, 2H) 6.91 (m, 2H) 6.80 (m, 1H) 4.26 (s, 2H) 3.94 (s, 3H) 3.74 (s, 3H)

### Example 47

### Methyl 2-(2-(2,5-dimethoxybenzyl)-1H-benzo[d]imidazol-1-yl)acetate

Example 3 is repeated to obtain 124 mg of the target compound (yield: 65%), except that 2-(2,5-dimethoxybenzyl)-1H-benzo[d]imidazole (150 mg, 0.56 mmol), sodium hydride (16 mg, 0.67 mmol) and methyl bromoacetate (67 μL, 0.73 mmol) are used.

¹H NMR (300 MHz, CDCl₃-d) δ ppm 7.78 (m, 1H) 7.25 (m, 3H) 7.18 (m, 1H) 6.83 (d, J=3.60 Hz) 6.76 (m, 2H) 4.83 (s, 2H) 4.29 (s, 2H) 3.93 (s, 3H) 3.67 (s, 3H) 3.59 (s, 3H)

### Example 48

### 2-(2-(2,5-dimethoxybenzyl)-1H-benzo[d]imidazol-1-yl)acetic acid

Example 4 is repeated to obtain 120 mg of the target compound (yield: 95%), except that methyl 2-(2-(2,5-dimethoxybenzyl)-1H-benzo[d]imidazol-1-yl)acetate (124 mg, 0.36 mmol) and 1N sodium hydroxide (1.09 mL, 1.09 mmol) are used.

¹H NMR (300 MHz, MeOD) δ ppm 7.80 (m, 1H) 7.75 (m, 1H) 7.62 (m, 2H) 7.05 (s, 1H) 6.97 (s, 2H) 5.40 (s, 2H) 4.51 (s, 2H) 3.80 (s, 3H) 3.72 (s, 3H)

### Example 49

### N-(3,5-di-tert-butylphenyl)-2-(2,5-dimethoxybenzyl)-1H-benzo[d]imidazol-1-yl)acetamide

Example 5 is repeated to obtain 27 mg of the target compound (yield: 86%), except that 2-(2-(2,5-dimethoxybenzyl)-1H-benzo[d]imidazol-1-yl)acetic acid (20 mg, 0.06 mmol), 3,5-di-tert-butylaniline (16 mg, 0.08 mmol), HATU (46 mg, 0.12 mmol) and TEA (42 μL, 0.30 mmol) are used.

¹H NMR (300 MHz, MeOD) δ ppm 7.61 (m, 1H) 7.41 (m, 1H) 7.37 (s, 2H) 7.24 (m, 2H) 7.20 (m, 1H) 6.84 (d, J=8.60, 1H) 6.74 (m, 2H) 5.02 (s, 2H) 4.29 (s, 2H) 3.76 (s, 3H) 3.61 (s, 3H) 1.29 (s, 18H)

### Example 50

### 2-(2-(2,5-dimethoxybenzyl)-1H-benzo[d]imidazol-1-yl)-N-(5-isopropyl-2-methylphenyl)acetamide

Example 5 is repeated to obtain 10 mg of the target compound (yield: 36%), except that 2-(2-(2,5-dimethoxybenzyl)-1H-benzo[d]imidazol-1-yl)acetic acid (20 mg, 0.06 mmol), 5-isopropyl-2-methylaniline (12 mg, 0.08 mmol), HATU (46 mg, 0.12 mmol) and TEA (42 μL, 0.30 mmol) are used.

¹H NMR (300 MHz, MeOD) δ ppm 7.66 (m, 1H) 7.47 (m, 1H) 7.30 (m, 2H) 7.16 (s, 1H) 7.11 (d, J=7.84, 1H) 7.01 (m, 1H) 6.92 (d, J=8.08, 1H) 6.76 (m, 2H) 5.13 (s, 2H) 4.32 (s, 2H) 3.81 (s, 3H) 3.64 (s, 3H) 2.83 (heptet, J=7.01 Hz, 1H) 2.12 (s, 3H) 1.22 (s, 3H) 1.20 (s, 3H)

### Example 51

### N-(3,5-dichlorophenyl)-2-(2-(2,5-dimethoxybenzyl)-1H-benzo[d]imidazol-1-yl)acetamide

Example 5 is repeated to obtain 19 g of the target compound (yield: 44%), except that 2-(2-(2,5-dimethoxybenzyl)-1H-benzo[d]imidazol-1-yl)acetic acid (30 mg, 0.09 mmol), 3,5-dichloroaniline (15 mg, 0.20 mmol), HATU (68 mg, 0.18 mmol) and TEA (63 μL, 0.45 mmol) are used.

¹H NMR (300 MHz, CDCl₃-d) δ ppm 7.94 (s, 1H) 7.70 (m, 1H) 7.27 (m, 2H) 7.22 (s, 2H) 7.02 (s, 1H) 6.80 (m, 1H) 6.70 (d, J=8.96, 1H) 6.60 (m, 1H) 4.92 (s, 2H) 4.24 (s, 2H) 3.76 (s, 3H) 3.60 (s, 3H)

### Example 52

### 2,5-dichloro-N-(4-chloro-2-iodophenyl)benzamide

Example 1 is repeated to obtain 700 mg of the target compound (yield: 83%), except that 4-chloro-2-iodoaniline (500 mg, 1.97 mmol) and 2,5-dichlorobenzoyl chloride (0.38 mg, 2.17 mmol) are used.

¹H NMR (300 MHz, DMSO-d₆) δ ppm 10.38 (s, 1H) 7.95 (d, J=8.49 Hz, 1H) 7.78 (s, 1H) 7.62 (s, 3H) 7.17 (dd, J=10.92 Hz, 1H)

### Example 53

### 5-chloro-2-(2,5-dichlorophenyl)-1H-benzo[d]imidazole

Example 2 is repeated to obtain 140 mg of the target compound (yield: 29%), except that 2,5-dichloro-N-(4-chloro-2-iodophenyl)benzamide (700 mg, 1.64 mmol), copper iodide (30 mg, 0.16 mmol), L-proline (36.8 mg, 0.32 mmol), sodium hydride (98 mg, 2.46 mmol), 30% aqueous ammonia (0.16 mL, 2.46 mmol) and acetic acid (7 mL) are used.

¹H NMR (300 MHz, MeOD) δ ppm 7.90 (s, 1H) 7.67 (brs, 1H) 7.65 (s, 1H) 7.62 (s, 1H) 7.57 (dd, J=11.14 Hz, 1H) 7.32 (dd, J=10.55 Hz, 1H)

### Example 54

### Methyl 2-(5-chloro-2-(2,5-dichlorophenyl)-1H-benzo[d]imidazol-1-yl)acetate and Methyl 2-(6-chloro-2-(2,5-dichlorophenyl)-1H-benzo[d]imidazol-1-yl)acetate

Example 3 is repeated to obtain 174 mg of the target compound (yield: 100%), except that 5-chloro-2-(2,5-dichlorophenyl)-1H-benzo[d]imidazole (130 mg, 0.44 mmol), sodium hydride (12.48 mg, 0.52 mmol) and methyl bromoacetate (53 μL, 0.57 mmol) are used.

¹H NMR (300 MHz, CDCl₃-d) δ ppm 7.82 (s, 1H) 7.74 (d, J=8.27 Hz, 1H) 7.57 (s, 2H) 7.45 (s, 4H) 7.34 (s, 2H) 7.29 (m, 1H) 7.24 (d, J=8.62 Hz, 1H) 4.76 (s, 2H) 4.74 (s, 2H) 3.71 (s, 3H) 3.70 (s, 3H)

### Example 55

### Methyl 2-(5-chloro-2-(2,5-dichlorophenyl)-1H-benzo[d]imidazol-1-yl)acetic acid and Methyl 2-(6-chloro-2-(2,5-dichlorophenyl)-1H-benzo[d]imidazol-1-yl)acetic acid

Example 4 is repeated to obtain 310 mg of the target compound (yield: 80%), except that methyl 2-(5-chloro-2-(2,5-dichlorophenyl)-1H-benzo[d]imidazol-1-yl)acetate and methyl 2-(6-chloro-2-(2,5-dichlorophenyl)-1H-benzo[d]imidazol-1-yl)acetate (400 mg, 1.19 mmol) and 1N sodium hydroxide (3.58 mL, 3.59 mmol) are used.

¹H NMR (300 MHz, DMSO-d₆) δ ppm 7.82 (dd, J=15.0 Hz, 1H) 7.70 (m, 3H) 7.60 (s, 1H) 7.35 (m, 1H) 4.91 (s, 2H)

### Example 56

### 2-(5-chloro-2-(2,5-dichlorophenyl)-1H-benzo[d]imidazol-1-yl)-N-(3,5-di-tert-butylphenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (40 mg, 0.11 mmol), 3,5-di-tert-butylaniline (35 mg, 0.17 mmol), HATU (84 mg, 0.22 mmol) and TEA (46 μL, 0.33 mmol) are allowed to react. The filtrate is concentrated under reduced pressure and the concentrate is purified by column chromatography (ether:MC=1:40). The resultant product is recrystallized with diethyl ether to obtain 14 mg of the target compound (yield: 35%).

¹H NMR (300 MHz, CDCl₃-d) δ ppm 7.90 (s, 1H) 7.64 (s, 1H) 7.51 (m, 2H) 7.41 (m, 2H) 7.23 (s, 1H) 7.18 (m, 2H) 7.06 (brs, 1H) 4.82 (s, 2H) 1.29 (s, 18H)

### Example 57

### 2-(6-chloro-2-(2,5-dichlorophenyl)-1H-benzo[d]imidazol-1-yl)-N-(3,5-di-tert-butylphenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (40 mg, 0.11 mmol), 3,5-di-tert-butylaniline (35 mg, 0.17 mmol), HATU (84 mg, 0.22 mmol) and TEA (46 μL, 0.33 mmol) are allowed to react. The filtrate is concentrated under reduced pressure and the concentrate is purified by column chromatography (ether:MC=1:40). The resultant product is recrystallized with diethyl ether to obtain 14 mg of the target compound (yield: 35%).

¹H NMR (300 MHz, CDCl₃-d) δ ppm 7.83 (d, J=8.70 Hz, 1H) 7.65 (s, 1H) 7.51 (m, 2H) 7.48 (s, 1H) 7.39 (d, J=10.20 Hz, 1H) 7.23 (s, 1H) 7.20 (s, 2H) 7.11 (brs, 1H) 4.80 (s, 2H) 1.30 (s, 18H)

### Example 58

### 2-(5-chloro-2-(2,5-dichlorophenyl)-1H-benzo[d]imidazol-1-yl)-N-(5-isopropyl-2-methylphenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (40 mg, 0.11 mmol), 5-isopropyl-2-methylaniline (35 mg, 0.17 mmol), HATU (84 mg, 0.22 mmol) and TEA (46 μL, 0.33 mmol) are allowed to react. The filtrate is concentrated under reduced pressure and the concentrate is purified by column chromatography (ether:MC=1:40). The resultant product is recrystallized with diethyl ether to obtain 20 mg of the target compound (yield: 45%).

¹H NMR (300 MHz, MeOD) δ ppm 7.92 (s, 1H) 7.67 (s, 3H) 7.64 (s, 1H) 7.42 (d, J=10.5 Hz, 1H) 7.09 (m, 2H) 7.02 (d, J=9.6 Hz, 2H) 5.09 (s, 2H) 2.83 (heptet, J=7.01 Hz, 1H) 2.02 (s, 3H) 1.22 (s, 3H) 1.20 (s, 3H)

### Example 59

### 2-(6-chloro-2-(2,5-dichlorophenyl)-1H-benzo[d]imidazol-1-yl)-N-(5-isopropyl-2-methylphenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (40 mg, 0.11 mmol), 5-isopropyl-e-methylaniline (35 mg, 0.17 mmol), HATU (84 mg, 0.22 mmol) and TEA (46 μL, 0.33 mmol) are allowed to react. The filtrate is concentrated under reduced pressure and the concentrate is purified by column chromatography (ether:MC=1:40). The resultant product is recrystallized with diethyl ether to obtain 20 mg of the target compound (yield: 45%).

¹H NMR (300 MHz, MeOD) δ ppm 7.82 (s, 1H) 7.74 (d, J=4.5 Hz, 1H) 7.71 (s, 3H) 7.39 (d, J=10.5 Hz, 1H) 7.12 (d, J=7.8 Hz, 1H) 7.09 (s, 1H) 7.02 (d, J=9.30 Hz, 1H) 5.09 (s, 2H) 2.83 (heptet, J=7.01 Hz, 1H) 2.11 (s, 3H) 1.22 (s, 3H) 1.20 (s, 3H)

### Example 60

### 2-(5-chloro-2-(2,5-dichlorophenyl)-1H-benzo[d]imidazol-1-yl)-N-(3,5-dichlorophenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (47 mg, 0.13 mmol), 3,5-dichloroaniline (32 mg, 0.2 mmol), HATU (99 mg, 0.20 mmol) and TEA (37 μL, 0.26 mmol) are allowed to react. The filtrate is concentrated under reduced pressure and the concentrate is purified by column chromatography (ether:MC=1:40). The resultant product is recrystallized with diethyl ether to obtain 10 mg of the target compound (yield: 33%).

¹H NMR (300 MHz, CDCl₃-d) δ ppm 7.88 (s, 1H) 7.59 (s, 1H) 7.51 (s, 2H) 7.40 (m, 1H) 7.32 (m, 3H) 7.18 (s, 1H) 7.14 (s, 1H) 4.80 (s, 2H)

### Example 61

### 2-(6-chloro-2-(2,5-dichlorophenyl)-1H-benzo[d]imidazol-1-yl)-N-(3,5-dichlorophenyl)acetamide

In the same manner as described in Example 5, a mixture of methyl 2-(5-chloro-2-(2,5-dichlorophenyl)-1H-benzo[d]imidazol-1-yl)acetic acid and methyl 2-(6-chloro-2-(2,5-dichlorophenyl)-1H-benzo[d]imidazol-1-yl)acetic acid (47 mg, 0.13 mmol), 3,5-dichloroaniline (32 mg, 0.2 mmol), HATU (99 mg, 0.20 mmol) and TEA (37 μL, 0.26 mmol) are allowed to react. The filtrate is concentrated under reduced pressure and the concentrate is purified by column chromatography (ether:MC=1:40). The resultant product is recrystallized with diethyl ether to obtain 10 mg of the target compound (yield: 33%).

¹H NMR (300 MHz, MeOD) δ ppm 7.74 (m, 2H) 7.65 (m, 3H) 7.51 (d, J=1.80 Hz, 2H) 7.40 (dd, J=10.50 Hz, 1H) 7.19 (m, 1H) 5.01 (s, 2H)

### Example 62

### 5-chloro-2-(2,6-dichlorobenzyl)-1H-benzo[d]imidazole

In the same manner as described in Example 40, 4-chlorobenzene-1,2-diamine (500 mg, 3.5 mmol), 2-(2,6-dichlorophenyl)acetic acid (863 mg, 4.21 mmol) and polyphosphoric acid (PPA, 2.6 g, 12.63 mmol) are used to obtain 1 g of the target compound (yield: 92%).

¹H NMR (300 MHz, DMSO-d₆) δ ppm 7.42 (m, 5H) 7.13 (m, 1H) 4.48 (s, 2H)

### Example 63

### Methyl 2-(5-chloro-2-(2,6-dichlorobenzyl)-1H-benzo[d]imidazol-1-yl)acetate and Methyl 2-(6-chloro-2-(2,6-dichlorobenzyl)-1H-benzo[d]imidazol-1-yl)acetate

Example 3 is repeated to obtain 380 mg of the target compound (yield: 95%), except that 5-chloro-2-(2,6-dichlorobenzyl)-1H-benzo[d]imidazole (400 mg, 1.28 mmol), sodium hydride (37 mg, 1.54 mmol) and methyl bromoacetate (130 μL, 1.41 mmol) are used.

¹H NMR (300 MHz, CDCl₃-d) δ ppm 7.73 (s, 1H) 7.64 (d, J=8.40 Hz, 1H) 7.39 (m, 4H) 7.22 (m, 6H) 4.95 (s, 2H) 4.93 (s, 2H) 4.52 (s, 2H) 4.51 (s, 2H) 3.79 (s, 3H) 3.77 (s, 3H)

### Example 64

### 2-(5-chloro-2-(2,6-dichlorobenzyl)-1H-benzo[d]imidazol-1-yl)acetic acid and 2-(6-chloro-2-(2,5-dichlorobenzyl)-1H-benzo[d]imidazol-1-yl)acetic acid

Example 4 is repeated to obtain 180 mg of the target compound (yield: 90%), except that a mixture of methyl 2-(5-chloro-2-(2,6-dichlorobenzyl)-1H-benzo[d]imidazol-1-yl)acetate and methyl 2-(6-chloro-2-(2,5-dichlorobenzyl)-1H-benzo[d]imidazol-1-yl)acetate (200 mg, 0.75 mmol) and 1N sodium hydroxide (2.25 mL, 2.25 mmol) are used.

¹H NMR (300 MHz, DMSO-d₆) δ ppm 13.36 (brs, 1H) 7.74 (s, 1H) 7.54 (m, 7H) 7.39 (dd, J=15.9 Hz, 2H) 7.23 (dd, J=10.5 Hz, 1H) 7.15 (dd, J=10.5 Hz, 1H) 5.28 (s, 4H) 4.46 (s, 2H) 4.44 (s, 2H)

### Example 65

### 2-(5-chloro-2-(2,6-dichlorobenzyl)-1H-benzo[d]imidazol-1-yl)-N-(5-isopropyl-2-methylphenyl)acetamide

A mixture of 2-(5-chloro-2-(2,6-dichlorobenzyl)-1H-benzo[d]imidazol-1-yl)acetic acid and 2-(6-chloro-2-(2,6-dichlorophenyl)-1H-benzo[d]imidazol-1-yl)acetic acid (500 mg, 1.35 mmol), 5-isopropyl-2-methylaniline (403 mg, 2.71 mmol), HATU (1.03 mg, 2.71 mmol) and TEA (380 μL, 2.71 mmol) are allowed to react. The filtrate is concentrated under reduced pressure and the concentrate is purified by column chromatography (ether:MC=1:40) to obtain 530 mg of the target compound (yield: 78%).

¹H NMR (300 MHz, DMSO-d₆) δ ppm 9.85 (brs, 1H) 7.77 (s, 1H) 7.52 (m, 3H) 7.38 (m, 1H) 7.32 (s, 1H) 7.15 (m, 2H) 7.00 (m, 1H) 5.33 (s, 2H) 4.53 (s, 2H) 2.82 (heptet, J=7.01 Hz, 1H) 2.22 (s, 3H) 1.16 (s, 3H) 1.15 (s, 3H)

### Example 66

### 2-(6-chloro-2-(2,6-dichlorobenzyl)-1H-benzo[d]imidazol-1-yl)-N-(5-isopropyl-2-methylphenyl)acetamide

A mixture of 2-(5-chloro-2-(2,6-dichlorobenzyl)-1H-benzo[d]imidazol-1-yl)acetic acid and 2-(6-chloro-2-(2,6-dichlorophenyl)-1H-benzo[d]imidazol-1-yl)acetic acid (500 mg, 1.35 mmol), 5-isopropyl-2-methylaniline (403 mg, 2.71 mmol), HATU (1.03 mg, 2.71 mmol) and TEA (380 μL, 2.71 mmol) are allowed to react. The filtrate is concentrated under reduced pressure and the concentrate is purified by column chromatography (ether:MC=1:40) to obtain 530 mg of the target compound (yield: 78%).

¹H NMR (300 MHz, CDCl₃-d) δ ppm 7.81 (s, 1H) 7.64 (s, 1H) 7.40 (d, J=8.1 Hz, 2H) 7.28 (m, 5H) 7.00 (dd, J=26.4 Hz, 2H) 6.85 (s, 1H) 5.06 (s, 2H) 4.64 (s, 2H) 2.87 (heptet, J=7.01 Hz, 1H) 1.72 (s, 3H) 1.27 (s, 3H) 1.24 (s, 3H)

### Example 67

### Methyl 4-((2-(2,6-dichlorobenzyl)-1H-benzo[d]imidazol-1-yl)methyl)benzoate

2-(2,6-dichlorobenzyl)-1H-benzo[d]imidazole (1.0 g, 3.61 mmol) and sodium hydride (347 mg, 14.44 mmol) are dissolved into DMF (20 mL) under nitrogen. At room temperature, the reaction mixture is agitated for 16 hours. After adding methyl 4-(bromomethyl)benzoate (0.99 g, 4.33 mmol) thereto, the reaction mixture is agitated for 16 hours at room temperature. The reaction progress and result are checked by TLC. After completing the reaction, water is added to the reaction mixture and is extracted with ethyl acetate. The organic layer is dried with dry MgSO₄, followed by filtering. The filtrate is concentrated under reduced pressure and the concentrate is recrystallized with diethyl ether to obtain 800 mg of the target compound (yield: 52%).

¹H NMR (300 MHz, CDCl₃-d) δ ppm 7.99 (d, J=8.40 Hz, 2H) 7.81 (m, 1H) 7.32 (m, 2H) 7.25 (m, 3H) 7.17 (m, 3H) 5.54 (s, 2H) 4.50 (s, 2H) 3.95 (s, 3H)

### Example 68

### 4-((2-(2,6-dichlorobenzyl)-1H-benzo[d]imidazole-1-yl)methyl)benzoic acid

Methyl 4-((2-(2,6-dichlorobenzyl)-1H-benzo[d]imidazol-1-yl)methyl)benzoate (100 mg, 0.75 mmol) is dissolved into THF (4 mL) under nitrogen. Lithium hydroxide (17 mg, 0.71 mmol) dissolved in water is added dropwise thereto gradually at room temperature and one or two drops of MeOH are further added thereto to allow sufficient mixing with the solvent. While the reaction mixture is agitated for 2-3 hours, the reaction progress and result are checked by TLC. After completing the reaction, the reaction mixture is concentrated under reduced pressure. Then, 1N HCl is added dropwise thereto gradually to perform acidification, and the resultant solid is filtered to obtain 80 mg of the target compound (yield: 83%).

¹H NMR (300 MHz, DMSO-d₆) δ ppm 7.93 (d, J=8.10 Hz, 2H) 7.68 (m, 2H) 7.54 (d, J=7.80, 2H) 7.41 (m, 5H) 5.87 (s, 2H) 4.69 (s, 2H)

### Example 69

### 4-((2-(2,6-dichlorobenzyl)-1H-benzo[d]imidazol-1-yl)methyl)-N-((1-ethylpyrrolidin-2-yl)methyl)benzamide

4-((2-(2,6-dichlorobenzyl)-1H-benzo[d]imidazol-1-yl)methyl)benzoic acid (100 mg, 0.24 mmol) is dissolved into methylene chloride and oxalyl chloride (42 μL, 0.48 mmol) is added dropwise thereto at room temperature. Then, one or two drops of dimethylformamide are added thereto as a catalyst and the reaction mixture is allowed to react for 2 hours at room temperature. The reaction progress is checked by TLC. When it is determined that the reaction is completed, the reaction mixture is concentrated under reduced pressure. The concentrate is dissolved into dry tetrahydrofuran, and then (1-ethylpyrrolidin-2-yl)methaneamine (31 μL, 0.26 mmol) and diisopropyl ethylamine (DIPEA, 86 μL, 0.60 mmol) are added dropwise thereto in turn. After the reaction mixture is allowed to react for 16 hours at room temperature, TLC is carried out to determine the completion of the reaction. The reaction mixture is concentrated under reduced pressure, diluted with methylene chloride, and extracted with water. Then, the combined organic layer is dried with dry sodium sulfate. The resultant product is concentrated under reduced pressure and subjected to column chromatography (MC:MeOH=10:1) to obtain 100 mg of the target compound (yield: 79%).

¹H NMR (300 MHz, CDCl₃-d) δ ppm 7.76 (dd, J=12.75 Hz, 3H) 7.30 (m, 2H) 7.24 (m, 3H) 7.14 (m, 3H) 7.04 (brs, 1H) 5.50 (s, 2H) 4.48 (s, 2H) 3.68 (m, 1H) 3.28 (m, 2H) 2.85 (m, 1H) 2.31 (brs, 1H) 2.25 (m, 2H) 1.89 (m, 1H) 1.70 (m, 3H) 1.13 (t, J=14.37 Hz, 3H)

### Example 70

### 4-((2-(2,6-dichlorobenzyl)-1H-benzo[d]imidazol-1-yl)methyl)-N-((1-methylpiperidin-4-yl)benzamide

Example 69 is repeated to obtain 5 mg of the target compound (yield: 4%), except that 4-((2-(2,6-dichlorobenzyl)-1H-benzo[d]imidazol-1-yl)methylbenzoic acid (50 mg, 0.12 mmol), oxalyl chloride (20 μL, 0.24 mmol), 1-methylpiperidin-4-amine (15 μL, 0.13 mmol), DIPEA (43 μL, 0.30 mmol) are used.

¹H NMR (300 MHz, MeOD) δ ppm 7.80 (d, J=8.40 Hz, 2H) 7.58 (m, 1H) 7.44 (m, 3H) 7.28 (m, 5H) 5.71 (s, 2H) 4.56 (s, 2H) 3.95 (m, 1H) 3.08 (d, J=12.0 Hz, 2H) 2.45 (m, 5H) 2.02 (m, 2H) 1.77 (m, 2H)

### Example 71

### 4-((2-(2,6-dichlorobenzyl)-1H-benzo[d]imidazol-1-yl)methyl)-N-(3-(dimethylamino)propyl)benzamide

Example 69 is repeated to obtain 30 mg of the target compound (yield: 24%), except that 4-((2-(2,6-dichlorobenzyl)-1H-benzo[d]imidazol-1-yl)methyl)benzoic acid (50 mg, 0.12 mmol), oxalyl chloride (20 μL, 0.24 mmol), N1,N1-dimethylpropan-1,3-diamine (16 μL, 0.13 mmol), DIPEA (43 μL, 0.30 mmol) are used.

¹H NMR (300 MHz, MeOD) δ ppm 7.80 (m, 2H) 7.56 (m, 1H) 7.41 (m, 3H) 7.25 (m, 5H) 5.68 (s, 2H) 4.54 (s, 2H) 3.44 (t, J=13.5 Hz, 2H) 2.82 (t, J=15.3 Hz, 2H) 2.60 (s, 6H) 1.93 (q, J=15.0 Hz, 2H)

### Example 72

### 4-((2-(2,6-dichlorobenzyl)-1H-benzo[d]imidazol-1-yl)methyl)-N-(3-pyrrolidin-1-yl)propyl)benzamide

4-((2-(2,6-dichlorobenzyl)-1H-benzo[d]imidazol-1-yl)methyl)benzoic acid (100 mg, 0.24 mmol), benzotriazol-1-yl-oxy-tris-(dimethylamino)-phosphonium hexafluorophosphate (BOP, 212 mg, 0.48 mmol) are dissolved into DMF (2 mL). DIPEA (52 μL, 0.36 mmol) and 3-(pyrrolidin-1-yl)propan-1-amine (35 μL, 0.27 mmol) are added dropwise thereto and the reaction mixture is allowed to react for 16 hours at room temperature. After determining the completion of the reaction by TLC, the reaction mixture is concentrated under reduced pressure. The reaction mixture is diluted with ethyl acetate, extracted with water and the combined organic layer is dried with dry sodium sulfate. Column chromatography (MC:MeOH=10:1) is carried out to obtain 47 mg of the target compound (yield: 3%).

¹H NMR (300 MHz, CDCl₃-d) δ ppm 7.81 (m, 2H) 7.69 (m, 1H) 7.33 (m, 2H) 7.27 (m, 4H) 7.20 (m, 2H) 5.55 (s, 2H) 4.50 (s, 2H) 3.51 (t, J=12.9 Hz, 2H) 3.39 (s, 4H) 2.60 (s, 6H) 1.93 (q, J=15.0 Hz, 2H)

### Example 73

### 4-((2-(2,6-dichlorobenzyl)-1H-benzo[d]imidazol-1-yl)methyl)-N-(3-(4-methylpiperazin-1-yl)propyl)benzamide

Example 72 is repeated to obtain 100 mg of the target compound (yield: 75%), except that 4-((2-(2,6-dichlorobenzyl)-1H-benzo[d]imidazol-1-yl)methyl)benzoic acid (100 mg, 0.24 mmol), BOP (212 mg, 0.48 mmol), DIPEA (52 μL, 0.36 mmol) and 3-(4-methylpiperazin-1-yl)propan-1-amine (30 μL, 0.27 mmol) are used.

¹H NMR (300 MHz, DMSO-d₆) δ ppm 8.49 (brs, 1H) 7.80 (d, J=8.1 Hz, 2H) 7.50 (d, J=8.1 Hz, 4H) 7.37 (m, 1H) 7.26 (m, 2H) 7.15 (m, 2H) 5.72 (s, 2H) 4.48 (s, 2H) 3.39 (brs, 2H) 3.17 (m, 8H) 2.39 (m, 2H) 2.29 (s, 3H) 1.66 (q, J=13.5 Hz, 2H)

### Example 74

### 4-((2-(2,6-dichlorobenzyl)-1H-benzo[d]imidazol-1-yl)methyl)-N-(3-(piperazin-1-yl)propyl)benzamide

Example 72 is repeated to obtain 37 mg of the target compound (yield: 27%), except that 4-((2-(2,6-dichlorobenzyl)-1H-benzo[d]imidazol-1-yl)methyl)benzoic acid (100 mg, 0.24 mmol), BOP (212 mg, 0.48 mmol), DIPEA (52 μL, 0.36 mmol) and 3-(4-methylpiperazin-1-yl)propan-1-amine (34 μL, 0.27 mmol) are used.

¹H NMR (300 MHz, MeOD) δ ppm 7.83 (m, 2H) 7.57 (m, 1H) 7.45 (m, 3H) 7.26 (m, 5H) 5.71 (s, 2H) 4.56 (s, 2H) 3.47 (t, J=13.2 Hz, 2H) 3.15 (brs, 2H) 3.06 (t, J=15.6 Hz, 4H) 3.06 (q, J=15.3 Hz, 2H) 1.84 (t, J=11.1 Hz, 4H) 1.31 (brs, 2H)

### Example 75

### 4-((2-(2,6-dichlorobenzyl)-1H-benzo[d]imidazol-1-yl)methyl)-N-(3-(piperazin-1-yl)ethyl)benzamide

Example 72 is repeated to obtain 37 mg of the target compound (yield: 27%), except that 4-((2-(2,6-dichlorobenzyl)-1H-benzo[d]imidazol-1-yl)methyl)benzoic acid (100 mg, 0.24 mmol), BOP (212 mg, 0.48 mmol), DIPEA (52 μL, 0.36 mmol) and 3-(4-methylpiperazin-1-yl)ethan-1-amine (38 μL, 0.27 mmol) are used.

¹H NMR (300 MHz, MeOD) δ ppm 7.85 (d, J=8.4 Hz, 2H) 7.59 (m, 1H) 7.45 (m, 3H) 7.29 (m, 5H) 5.73 (s, 2H) 4.57 (s, 2H) 3.75 (t, J=12.0 Hz, 2H) 3.33 (m, 6H) 1.88 (brs, 4H) 1.72 (brs, 2H)

### Example 76

### 4-((2-(2,6-dichlorobenzyl)-1H-benzo[d]imidazol-1-yl)methyl)-N-(3-morpholinopropyl)benzamide

Example 72 is repeated to obtain 30 mg of the target compound (yield: 25%), except that 4-((2-(2,6-dichlorobenzyl)-1H-benzo[d]imidazol-1-yl)methyl)benzoic acid (100 mg, 0.24 mmol), BOP (212 mg, 0.48 mmol), DIPEA (52 μL, 0.36 mmol) and 3-morpholinopropan-1-amine (36 μL, 0.27 mmol) are used.

¹H NMR (300 MHz, MeOD) δ ppm 8.09 (s, 1H) 7.75 (m, 3H) 7.25 (m, 5H) 7.15 (m, 3H) 5.51 (s, 2H) 4.47 (s, 2H) 3.68 (t, J=9.0 Hz, 4H) 3.55 (t, J=17.1 Hz, 2H) 2.30 (m, 6H) 1.80 (q, J=24.6 Hz, 2H)

### Example 77

### 4-((2-(2,6-dichlorobenzyl)-1H-benzo[d]imidazol-1-yl)methyl)-N,N-dimethylbenzamide

Example 72 is repeated to obtain 15 mg of the target compound (yield: 28%), except that 4-((2-(2,6-dichlorobenzyl)-1H-benzo[d]imidazol-1-yl)methyl)benzoic acid (50 mg, 0.12 mmol), BOP (106 mg, 0.24 mmol), triethylamine (TEA, 50 μL, 0.36 mmol) and dimethylamine hydrochloride (24 mg, 0.30 mmol) are used.

¹H NMR (400 MHz, MeOD) δ ppm 7.88 (m, 1H) 7.75 (m, 1H) 7.65 (m, 2H) 7.54 (d, J=8.56 Hz, 2H) 7.45 (m, 3H) 7.34 (d, J=8.32 Hz, 2H) 5.95 (s, 2H) 5.03 (s, 2H) 3.11 (s, 3H) 2.98 (s, 3H)

### Example 78

### 2-(1H-benzo[d]imidazol-2-yl)acetic acid

2-(1H-benzo[d]imidazol-2-yl)acetonitrile (1 g, 6.36 mmol) is dissolved into ethanol. 4M sodium hydride (4.77 mL, 19.08 mmol) is added dropwise thereto gradually. The reaction mixture is warmed to 80° C. and allowed to react under reflux for 8 hours. After determining the completion of the reaction by TLC, the reaction mixture is concentrated under reduced pressure. The reaction mixture is acidified with 1N hydrochloric acid and the resultant solid is filtered to obtain 700 mg of the target compound (yield: 62%).

¹H NMR (300 MHz, DMSO-d₆) δ ppm 7.44 (m, 2H) 7.10 (m, 2H) 2.48 (s, 2H)

### Example 79

### 2-(1H-benzo[d]imidazole-2-yl)-N-(5-isopropyl-2-methylphenyl)acetamide

2-(1H-benzo[d]imidazole-2-yl)acetic acid (100 mg, 0.57 mmol), O-(7-azabenzotriazol-1-yl)-M,M,N′,N′-tetramethyluronium hexafluorophosphate (HATU, 433 mg, 1.14 mmol) are dissolved into acetonitrile (7 mL). Triethylamine (TEA, 160 μL, 1.14 mmol) is added thereto and 5-isopropyl-2-methylaniline (127 mg, 0.85 mmol) is further added dropwise thereto. After the reaction mixture is allowed to react for 16 hours at room temperature, TLC is performed to determine the completion of the reaction. The reaction mixture is concentrated under reduced pressure and extracted with water and ethyl acetate. The organic layer is dried with dry sodium sulfate. Column chromatography (EA:n-Hex=1:2) is carried out to obtain 170 mg of the target compound (yield: 81%).

¹H NMR (400 MHz, DMSO-d₆) δ ppm 12.37 (s, 1H) 9.91 (s, 1H) 7.56 (d, J=7.28 Hz, 1H) 7.47 (d, J=7.16 Hz, 1H) 7.43 (s, 1H) 7.14 (m, 3H) 6.95 (d, J=7.64 Hz, 1H) 4.02 (s, 2H) 2.82 (q, J=7.01 Hz, 1H) 2.20 (s, 2H) 1.17 (s, 3H) 1.15 (s, 3H)

### Example 80

### Methyl 4-((2-(2-(5-isopropyl-2-methylphenylamino)-2-oxoethyl)-1H-benzo[d]imidazol-1-yl)methyl)benzoate

2-(1H-benzo[d]imidazol-2-yl)-N-(5-isopropyl-2-methylphenyl)acetamide (100 mg, 0.33 mmol) and methyl 4-(bromomethyl)benzoate (89 mg, 0.39 mmol), diisopropylethylamine (DIPEA, 51 μL, 0.36 mmol) are mixed and the reaction mixture is warmed to 80° C. and allowed to react under reflux for 6 hours. After determining the completion of the reaction by TLC, the reaction mixture is concentrated under reduced pressure. Column chromatography (EA) is carried out to obtain 100 mg of the target compound (yield: 90%).

¹H NMR (400 MHz, CDCl₃-d) δ ppm 10.35 (s, 1H) 7.97 (d, J=8.32 Hz, 2H) 7.83 (s, 1H) 7.77 (d, J=7.24 Hz, 1H) 7.30 (m, 3H) 7.11 (m, 3H) 6.93 (d, J=7.80 Hz, 1H) 5.48 (s, 2H) 3.96 (s, 2H) 3.89 (s, 3H) 2.87 (q, J=7.01 Hz, 1H) 2.30 (s, 3H) 1.23 (s, 3H) 1.21 (s, 3H)

### Example 81

### 4-((2-(2-(5-isopropyl-2-methylphenylamino)-2-oxoethyl)-1H-benzo[d]imidazol-1-yl)methyl)benzoic acid

Example 68 is repeated to obtain 20 mg of the target compound (yield: 21%), except that methyl 4-((2-(2-(5-isopropyl-2-methylphenylamino)-2-oxoethyl)-1H-benzo[d]imidazol-1-yl)methyl)benzoate (100 mg, 0.22 mmol), and lithium hydroxide (150 mg, 0.66 mmol) are used.

¹H NMR (400 MHz, DMSO-d₆) δ ppm 12.94 (brs, 1H) 9.89 (s, 1H) 7.99 (d, J=8.30 Hz, 2H) 7.69 (d, J=7.19 Hz, 1H) 7.45 (m, 1H) 7.27 (m, 4H) 7.11 (d, J=7.86 Hz, 1H) 6.95 (dd, J=7.76 Hz, 1H) 5.73 (s, 2H) 4.24 (s, 2H) 2.78 (q, J=7.01 Hz, 1H) 2.18 (s, 3H) 1.13 (s, 3H) 1.11 (s, 3H)

### Example 82

### N-((1-ethylpyrrolidin-2-yl)methyl)-4-((2-(2-(5-isopropyl-2-methylphenylamino)-2-oxoethyl)-1H-benzo[d]imidazol-1-yl)methyl)benzamide

Example 72 is repeated to obtain 21 mg of the target compound (yield: 84%), except that methyl 4-((2-(2-(5-isopropyl-2-methylphenylamino)-2-oxoethyl)-1H-benzo[d]imidazol-1-yl)methyl)benzoic acid (20 mg, 0.045 mmol), BOP (40 mg, 0.09 mmol), TEA (7 μL, 0.05 mmol) and (1-ethylpyrrolidin-2-yl)methaneamine (7 μL, 0.05 mmol) are used.

¹H NMR (400 MHz, CDCl₃-d) δ ppm 10.53 (brs, 1H) 8.07 (s, 1H) 7.77 (m, 4H) 7.29 (m, 3H) 7.09 (m, 3H) 6.91 (d, J=7.6 Hz, 1H) 5.47 (s, 2H) 3.96 (s, 2H) 3.71 (m, 4H) 3.35 (m, 1H) 3.04 (m, 2H) 2.81 (m, 1H) 2.29 (s, 3H) 2.22 (m, 2H) 2.05 (m, 2H) 1.90 (s, 2H) 1.20 (s, 3H) 1.18 (s, 3H)

### Example 83

### N-(3-dimethylamino)propyl)-4-((2-(2-(5-isopropyl-2-methylphenylamino)-2-oxoethyl)-1H-benzo[d]imidazol-1-yl)methyl)benzamide

Example 72 is repeated to obtain 15 mg of the target compound (yield: 50%), except that 4-((2-(2-(5-isopropyl-2-methylphenylamino)-2-oxoethyl)-1H-benzo[d]imidazol-1-yl)methyl)benzoic acid (40 mg, 0.09 mmol), BOP (80 mg, 0.18 mmol), TEA (25 μL, 0.18 mmol) and N1,N1-dimethylpropan-1,3-diamine (10 μL, 0.10 mmol) are used.

¹H NMR (300 MHz, DMSO-d₆) δ ppm 7.83 (d, J=8.10 Hz, 2H) 7.76 (d, J=8.10 Hz, 1H) 7.40 (m, 5H) 7.20 (s, 1H) 7.14 (d, J=8.1 Hz, 1H) 7.02 (d, J=8.1 Hz, 1H) 5.79 (s, 2H) 4.48 (s, 2H) 3.49 (m, 4H) 3.13 (t, J=15.6 Hz, 2H) 2.87 (m, 2H) 2.20 (s, 3H) 2.00 (m, 4H) 1.79 (m, 2H) 1.43 (m, 1H) 1.28 (m, 1H) 1.21 (s, 3H) 1.19 (s, 3H)

### Example 84

### 4-((2-(2-(5-isopropyl-2-methylphenylamino)-2-oxoethyl)-1H-benzo[d]imidazol-1-yl)methyl-N-(3-(piperidin-1-yl)propylbenzamide

Example 72 is repeated to obtain 48 mg of the target compound (yield: 90%), except that 4-((2-(2-(5-isopropyl-2-methylphenylamino)-2-oxoethyl)-1H-benzo[d]imidazol-1-yl)methyl)benzoic acid (40 mg, 0.09 mmol), BOP (80 mg, 0.18 mmol), TEA (25 μL, 0.18 mmol) and 3-(piperidin-1-yl)propane-1-amine (14 μL, 0.10 mmol) are used.

¹H NMR (300 MHz, DMSO-d₆) δ ppm 7.83 (d, J=8.10 Hz, 2H) 7.76 (d, J=8.10 Hz, 1H) 7.40 (m, 5H) 7.20 (s, 1H) 7.14 (d, J=8.1 Hz, 1H) 7.02 (d, J=8.1 Hz, 1H) 5.79 (s, 2H) 4.48 (s, 2H) 3.49 (m, 4H) 3.13 (t, J=15.6 Hz, 2H) 2.87 (m, 2H) 2.20 (s, 3H) 2.00 (m, 4H) 1.79 (m, 2H) 1.43 (m, 1H) 1.28 (m, 1H) 1.21 (s, 3H) 1.19 (s, 3H)

### Example 85

### 1H-benzo[d]imidazole-2-thiol

Benzene-1,2-diamine (2 g, 18.49 mmol) is dissolved into ethanol (20 mL). After adding carbon disulfide (4 mL, 66.6 mmol) thereto, the reaction mixture is warmed to 60° C. and allowed to react under reflux for 5 hours. After determining the completion of the reaction by TLC, the reaction mixture is cooled to room temperature. The resultant solid is filtered while being washed with ethanol to obtain 2.8 g of the target compound (yield: 99%).

¹H NMR (300 MHz, DMSO-d₆) δ ppm 12.54 (s, 1H) 7.12 (m, 2H)

### Example 86

### Methyl 4-((1H-benzo[d]imidazol-2-ylthio)methyl)benzoate

1H-benzo[d]imidazole-2-thiol (100 mg, 0.67 mmol) is dissolved into acetone (3 mL). Potassium carbonate (185 mg, 1.34 mmol) dissolved in water is added thereto and the reaction mixture is agitated at room temperature for 30 minutes. After adding methyl 4-(bromomethyl)benzoate (169 mg, 0.74 mmol) thereto, the reaction mixture is warmed to 50° C. and allowed to react under reflux for 2 hours. After determining the completion of the reaction by TLC, the reaction mixture is concentrated under reduced pressure. The reaction mixture is diluted with ethyl acetate and extracted with water. Then, the organic layer is dried with dry sodium sulfonate. Column chromatography (EA:n-Hex=1:4) is carried out to obtain 212 mg of the target compound (yield: 99%).

¹H NMR (400 MHz, CDCl₃-d) δ ppm 8.94 (s, 1H) 7.98 (d, J=8.4 Hz, 2H) 7.73 (d, J=7.6 Hz, 1H) 7.48 (d, J=8.0 Hz, 2H) 7.34 (m, 1H) 7.24 (m, 2H) 4.62 (s, 2H) 3.92 (s, 3H)

### Example 87

### 4-((1H-benzo[d]imidazol-2-ylthio)methyl)benzoic acid

Example 68 is repeated to obtain 60 mg of the target compound (yield: 63%), except that methyl 4-((1H-benzo[d]imidazole-2-ylthio)methyl)benzoate (100 mg, 0.35 mmol) and lithium hydroxide (25 mg, 1.06 mmol) are used.

¹H NMR (400 MHz, DMSO-d₆) δ ppm 7.88 (d, J=8.4 Hz, 2H) 7.61 (m, 4H) 7.35 (m, 2H) 4.81 (s, 2H)

### Example 88

### 4-((1H-benzo[d]imidazol-2-ylthio)methyl)-N-((1-ethylpyrrolidin-2-yl)methyl)benzamide

Example 72 is repeated to obtain 40 mg of the target compound (yield: 57%), except that 4-((1H-benzo[d]imidazol-2-ylthio)methyl)benzoic acid (50 mg, 0.18 mmol), BOP (159 mg, 0.36 mmol), TEA (51 μL, 0.36 mmol) and (1-ethylpyrrolidin-2-yl)methaneamine (29 μL, 0.20 mmol) are used.

¹H NMR (300 MHz, MeOD) δ ppm 7.79 (d, J=8.1 Hz, 2H) 7.50 (m, 4H) 7.20 (m, 2H) 4.55 (s, 2H) 3.82 (m, 1H) 3.63 (m, 4H) 3.15 (m, 2H) 2.02 (m, 4H) 1.38 (t, J=14.4 Hz, 3H)

### Example 89

### 2-bromo-N-(5-isopropyl-2-methylphenyl)acetamide

2-bromoacetic acid (100 mg, 0.72 mmol) and O-(7-azabenzotriazol-1-yl)-N,N,N′,N′-tetramethyluronium hexafluorophosphate (HATU, 418 mg, 1.10 mmol) are dissolved into acetonitrile (4 mL). Triethylamine (TEA, 155 μL, 1.10 mmol) is added thereto and 5-isopropyl-2-methylaniline (83 mg, 0.55 mmol) is added dropwise thereto. The reaction mixture is allowed to react at room temperature for 16 hours, and TLC is performed to determine the completion of the reaction. The reaction mixture is concentrated under reduced pressure and extracted with water and ethyl acetate. The organic layer is dried with dry sodium sulfonate. Column chromatography (ether:n-Hex=1:2) is carried out to obtain 170 mg of the target compound (yield: 81%).

¹H NMR (300 MHz, CDCl₃-d) δ ppm 8.23 (s, 1H) 7.79 (s, 1H) 7.20 (d, J=9.9 Hz, 1H) 7.04 (dd, J=6.9 Hz, 1H) 4.28 (s, 1H) 2.95 (q, J=7.01 Hz, 1H) 2.31 (s, 3H) 1.32 (s, 3H) 1.29 (s, 3H)

### Example 90

### N-((1-ethylpyrrolidin-2-yl)methyl)-4-((1-(2-(5-isopropyl-2-methylphenylamino)-2-oxoethyl)-1H-benzo[d]imidazol-2-ylthio)methyl)benzamide

4-((1H-benzo[d]imidazol-2-ylthio)methyl)-N-((1-ethylpyrrolidin-2-yl)methyl)benzamide (152 mg, 0.39 mmol) and potassium carbonate (50 mg, 0.36 mmol) are dissolved into DMF (3 mL). 2-bromo-N-(5-isopropyl-2-methylphenyl)acetamide (90 mg, 0.33 mmol) is added thereto and the reaction mixture is agitated at room temperature for 16 hours. After determining the completion of the reaction by TLC, the reaction mixture is concentrated under reduced pressure. The reaction mixture is extracted with water and ethyl acetate, and the organic layer is dried with dry Na₂SO₄. Column chromatography (CDCl₃: MeOH:H₂O:NH₄OH=80:20:1:1) is carried out to obtain 80 mg of the target compound (yield: 41%).

¹H NMR (300 MHz, DMSO-d₆) δ ppm 9.76 (s, 1H) 8.38 (brs, 1H) 7.75 (d, J=7.8 Hz, 2H) 7.59 (m, 1H) 7.52 (m, 3H) 7.26 (s, 1H) 7.20 (m, 2H) 7.12 (d, J=8.1 Hz, 1H) 6.97 (d, J=7.5 Hz, 1H) 5.07 (s, 2H) 4.65 (s, 2H) 3.08 (brs, 2H) 2.79 (m, 3H) 2.18 (m, 6H) 1.72 (m, 4H) 1.14 (s, 3H) 1.12 (s, 3H) 1.06 (t, J=6.6 Hz, 3H)

### Example 91

### 4-((1H-benzo[d]imidazol-2-ylthio)methyl)-N-(3-(dimethylamino)propyl)benzamide

Example 72 is repeated to obtain 50 mg of the target compound (yield: 52%), except that 4-((1H-benzo[d]imidazol-2-ylthio)methyl)benzoic acid (70 mg, 0.26 mmol), BOP (230 mg, 0.52 mmol), TEA (73 μL, 0.52 mmol) and N1,N1-dimethylpropane-1,3-diamine (29 μL, 0.28 mmol) are used.

¹H NMR (300 MHz, MeOD) δ ppm 7.74 (m, 2H) 7.48 (m, 4H) 7.21 (m, 2H) 4.55 (s, 2H) 3.46 (m, 2H) 2.76 (m, 2H) 2.58 (s, 6H) 1.91 (m, 2H)

### Example 92

### N-(3-(dimethylamino)propyl)-4-((1-(2-(5-isopropyl-2-methylphenylamino)-2-oxoethyl)-1H-benzo[d]imidazol-2-ylthio)methyl)benzamide

Example 90 is repeated to obtain 11 mg of the target compound (yield: 20%), except that 4-((1H-benzo[d]imidazol-2-ylthio)methyl)-N-((1-ethylpyrrolidin-2-yl)methyl)benzamide (50 mg, 0.095 mmol), potassium carbonate (14 mg, 0.10 mmol) and 2-bromo-N-(5-isopropyl-2-methylphenyl)acetamide (27 mg, 0.10 mmol) are used.

¹H NMR (300 MHz, MeOD) δ ppm 7.71 (d, J=8.4 Hz, 3H) 7.64 (m, 1H) 7.45 (m, 3H) 7.29 (m, 3H) 7.21 (s, 1H) 7.13 (d, J=7.8 Hz, 1H) 7.04 (m, 1H) 5.07 (s, 2H) 4.57 (s, 2H) 3.39 (m, 2H) 2.65 (m, 1H) 2.56 (m, 2H) 2.47 (s, 6H) 2.19 (s, 3H) 1.88 (m, 2H) 1.21 (s, 3H) 1.19 (s, 3H)

### Example 93

### 4-((1-(2-(5-isopropyl-2-methylphenylamino)-2-oxoethyl)-1H-benzo[d]imidazol-2-ylthio)methyl)-N-(3-(piperidin-1-yl)propyl)benzamide

Example 90 is repeated to obtain 9 mg of the target compound (yield: 20%), except that 4-((1H-benzo[d]imidazol-2-ylthio)methyl)-N-((1-ethylpyrrolidin-2-yl)methyl)benzamide (50 mg, 0.095 mmol), potassium carbonate (14 mg, 0.10 mmol) and 2-bromo-N-(5-isopropyl-2-methylphenyl)acetamide (27 mg, 0.10 mmol) are used.

¹H NMR (400 MHz, MeOD) δ ppm 7.74 (d, J=8.24 Hz, 2H) 7.64 (dd, J=8.76 Hz, 1H) 7.47 (m, 3H) 7.29 (m, 2H) 7.21 (d, J=1.6 Hz, 1H) 7.14 (d, J=7.88 Hz, 1H) 7.03 (d, J=7.88 Hz, 1H) 5.08 (s, 2H) 4.58 (s, 2H) 3.47 (m, 4H) 3.10 (t J=15.52 Hz, 2H) 2.84 (m, 3H) 2.22 (s, 3H) 2.00 (m, 4H) 1.76 (m, 3H) 1.21 (m, 1H) 1.21 (s, 3H) 1.19 (s, 3H)

### Preparation Example

Meanwhile, the compound represented by Formula 1 according to the present invention may be formulated into various forms as desired. Hereinafter, some formulation examples containing the compound represented by Formula 1 are described, but the present invention is not limited thereto.

### Formulation Example 1

### Tablet (Direct Pressing)

5.0 mg of an active ingredient is sieved, and then mixed with 14.1 mg of lactose, 0.8 mg of crospovidone USNG and 0.1 mg of magnesium stearate, followed by pressing to provide tablets.

### Formulation Example 2

### Tablet (Wet Granulation)

5.0 mg of an active ingredient is sieved, and then mixed with 16.0 mg of lactose and 4.0 mg of starch. After dissolving 0.3 mg of polysorbate 80 into pure water, an adequate amount of the resultant solution is added to the above mixture and fine granulation is carried out. After drying, the fine granules are sieved and mixed with 2.7 mg of colloidal silicon dioxide and 2.0 mg of magnesium stearate. The fine granules are subjected to pressing to provide tablets.

### Formulation Example 3

### Powder and Capsule

5.0 mg of an active ingredient is sieved, and then mixed with 14.8 mg of lactose, 10.0 mg of polyvinyl pyrrolidone and 0.2 mg of magnesium stearate. The resultant mixture is filled into No. 5 gelatin capsules by using an adequate apparatus.

### Formulation Example 4

### Injection Formulation

In addition to 100 mg of an active ingredient, 180 mg of mannitol, 26 mg of Na₂HPO₄12H₂O and 2974 mg of distilled water are used to provide injection formulations.

Meanwhile, the compound represented by Formula 1 according to the present invention is evaluated through a test to determine recovery of mitochondrial functions damaged by amyloid beta, as described in the following Test Example. After the test, the results are shown in terms of the percentages of mitochondrial function disorder caused by amyloid beta. The degree of mitochondrial function disorder caused by amyloid beta is taken as 0% and the normal condition in the absence of amyloid beta is shown as 100%.

### Test Example

### Example 1

### Effect of Improvement in Mitochondrial Function Damaged by Amyloid Beta

To a transparent 96-well plate, HT-22 cells are inoculated in a number of 30,000 per well one day before the test. On the next day, JC-1 dye (Stratagen, USA) is diluted with Opti-MEM containing no phenol red to a concentration of 7.5 μM, and the resultant dilution is used to treat the cells for 1 hour and 15 minutes. The dyed cells are washed twice with phosphate buffer solution (PBS), followed by treatment with each compound to a final concentration of 5 μM per well. Then, the cells are cultured at 37° C. for 10 minutes. Particularly, for each drug, two wells are treated with amyloid beta (5 μM) and the other two wells are treated with the vehicle control (5 μM DMSO). Then, the cells are cultured at 37° C. for 3 hours. The fluorescence intensity of each well is read by using a fluorescence plate reader at 485/535 nm (green/J-monomer) and 560/595 nm (red/J-aggregate). Then, the ratio of green:red is calculated and the results are normalized by taking the change of the ratio between addition of amyloid beta and no-addition of amyloid beta in the vehicle control non-treated with drugs as 100%.

The following Table 1 shows the effect of improvement in mitochondrial membrane potentials dropped by amyloid beta after treatment with the compounds in terms of calculated percentages.

TABLE 1

| | Improvement in |
|---|---|
| | Membrane |
| Test | Potential (%) |
| Compounds | (5 μM) |
| Compound 1 | 41 |
| Compound 4 | 91 |
| Compound 5 | 91 |
| Compound 7 | 41 |
| Compound 12 | 38 |
| Compound 13 | 86 |
| Compound 14 | 73 |
| Compound 15 | 69 |
| Compound 16 | 65 |
| Compound 17 | 54 |
| Compound 18 | 68 |
| Compound 19 | 81 |
| Compound 21 | 57 |
| Compound 23 | 74 |
| Compound 24 | 42 |
| Compound 25 | 87 |
| Compound 26 | 60 |
| Compound 27 | 77 |
| Compound 32 | 90 |
| Compound 33 | 81 |
| Compound 34 | 82 |
| Compound 35 | 79 |
| Compound 36 | 86 |
| Compound 37 | 90 |
| Compound 38 | 89 |
| Compound 39 | 76 |
| Compound 41 | 68 |
| Compound 42 | 68 |
| Compound 43 | 70 |
| Compound 44 | 80 |
| Compound 45 | 59 |
| Compound 46 | 67 |
| Compound 47 | 73 |
| Cyclosporin A | 55 |

### Test Example 2

### Recovery of ATP Production Capability Reduced by Amyloid Beta

To a transparent 96-well plate, HT-22 cells are inoculated in a number of 10,000 per well one day before the test, followed by treatment with each compound to a final concentration of 5 μM per well. The cells are cultured at 37° C. for 10 minutes. Particularly, for each drug, two wells are treated with amyloid beta (5 μM) and the other two wells are treated with the vehicle control (5 μM DMSO). After the treatment, the cells are cultured at 37° C. for 7 hours. The cells treated with each drug are washed twice with PBS, lysed with Triton X-100 (1% in TSBT buffer) and agitated at room temperature for 5 minutes. BSA reagent is used to determine the protein amount of each well, and the same amount of protein taken from each well is added to a white 96-well plate. Then, potassium iodide (Molecular Probe, USA) for ATP determination containing d-luciferin and luciferase is added thereto and emission peaks generated from each of the wells are determined. After the determination, the peaks are normalized by taking the vehicle control to which amyloid beta is added without treatment with any drug as 0%.

TABLE 2

| | ATP Production Capability Recovery |
|---|---|
| Test Compounds | (%) (5 μM) |
| Compound 16 | 55 |
| Compound 17 | 46 |
| Compound 18 | 94 |
| Compound 23 | 95 |
| Compound 25 | 69 |
| Compound 32 | 51 |
| Compound 34 | 85 |
| Compound 37 | 64 |

As can be seen from the foregoing, the benzimidazole derivative represented by Formula 1 according to the present invention or a pharmaceutically acceptable salt thereof shows high activity as a neuro-protective agent effective for mitochondria, and thus is useful as an agent for treating diseases, such as Alzheimer's disease, Parkinson's disease, Huntington's disease, ischemic brain diseases, diabetes, schizophrenia, etc.

While the present invention has been described with respect to the specific embodiments, it will be apparent to those skilled in the art that various changes and modifications may be made without departing from the spirit and scope of the invention as defined in the following claims.

1. A compound selected from benzimidazole derivatives represented by the following Formula 1 and pharmaceutically acceptable salts thereof: 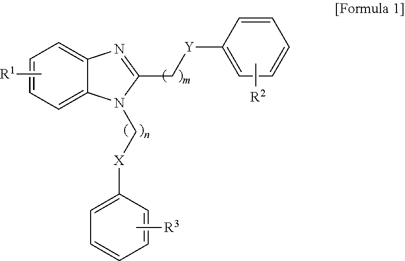
wherein X and Y are the same or different from each other and each represents a single bond or thiomethylene group, —C(O)NH— or —NHC(O); R¹ represents a hydrogen atom or halogen atom; R² and R³ are the same or different from each other and each represents a hydrogen atom, halogen atom, C₁-C₆ alkyl group, C₁-C₆ alkoxy group, phenoxy group, biphenyloxy group or —C(O)NR⁴R⁵; R⁴ and R⁵ are the same or different from each other and each represents a hydrogen atom, C₁-C₆ alkyl group or —(CH₂)l-NR⁶R⁷ (wherein R⁶ and R⁷ are the same or different from each other and each represents a hydrogen atom or C₁-C₆ alkyl group, or R⁶ and R⁷ represent a heteroalicyclic group selected from pyrrolidinyl, piperidinyl, piperazinyl and morpholino formed when they are bound to each other, and the heteroalicyclic group is substituted or non-substituted with a C₁-C₆ alkyl group); and l, m and n represent an integer of 0 to 6, with the proviso that when X and Y represent single bonds, R³ represents —C(O)NH—(CH₂)ₗ—NR⁶R⁷ (wherein R⁶ and R⁷ are bound to each other to form a heteroaliphatic cyclic group selected from the group consisting of pyrrolidinyl, piperidinyl, piperazinyl and morpholino, and the heteroaliphatic cyclic group is substituted or non-substitute with C₁-C₆ alkyl).

2. The compound according to claim 1, wherein X and Y are the same or different from each other, and each represents a single bond, thiomethylene group, —C(O)NH— or —NHC(O)—;
R¹ represents a hydrogen atom or chloro;
R² represents a hydrogen atom, chloro, methyl, ethyl, isopropyl, or methoxy group;
R³ represents chloro, fluoro, methyl, isopropyl, tert-butyl, biphenyloxy, aminocarbonyl, (methylamino)carbonyl, (dimethylamino)carbonyl, N-(aminopropyl)amide, N-[3-(methylamino)propyl]amide, N-[3-(dimethylamino)propyl]amide, N-[3-(pyrrolidine-1-yl)propyl]amide, N-[1-(ethylpyrrolidie-2-yl)methyl]amide, N-[2-(piperidien-1-yl)ethyl]amide, N-[3-(piperidine-1-yl)propyl]amide, N-(1-methylpiperidien-4-yl)amide, N-[3-(4-methylpiperazine-1-yl)propyl]amide, or N-(3-morpholinopropyl)amide group; and
m and n are the same or different from each other, and each represents an integer of 0, 1 or 2,
with the proviso that when X and Y represent single bonds, R³ represents N-[3-(pyrrolidin-1-yl)propyl]amide group, N-[1-(ethylpyrrolidin-2-yl)methyl]amide group, N-[2-(piperidin-1-yl)ethyl]amide group, N-[3-(piperidin-1-yl)propyl]amide group, N-(1-methylpiperidin-4-yl)amide group, N-[3-(4-methylpiperazin-1-yl)propyl]amide group, or N-(3-morpholinopropyl)amide group

3. The compound according to claim 1, which is a compound represented by the following Formula 1a: 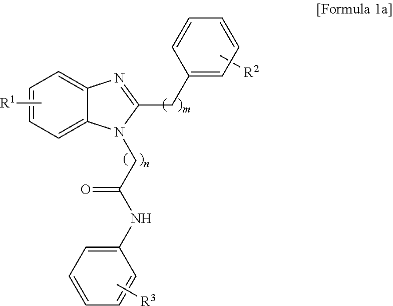
(wherein each of R¹, R², R³, m and n is the same as defined in claim 1).

4. The compound according to claim 1, which is a compound represented by the following Formulae 1b: 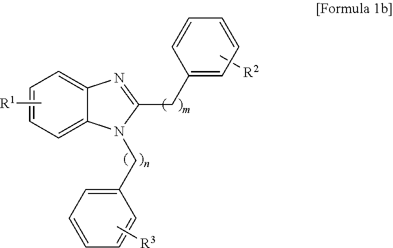
(wherein each of R¹, R², m and n is the same as defined in claim 1, and R³ represents —C(O)NH—(CH₂)ₗ—NR⁶R⁷ (wherein R⁶ and R⁷ are bound to each other to form a heteroaliphatic cyclic group selected from the group consisting of pyrrolidinyl, piperidinyl, piperazinyl and morpholino, and the heteroaliphatic cyclic group is substituted or non-substitute with C₁-C₆ alkyl)).

5. The compound according to claim 1, which is a compound represented by the following Formula 1c: 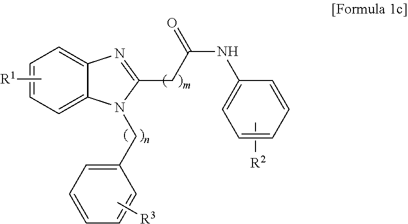
(wherein each of R¹, R², R³, m and n is the same as defined in claim 1).

6. The compound according to claim 1, which is a compound represented by the following Formula 1d: 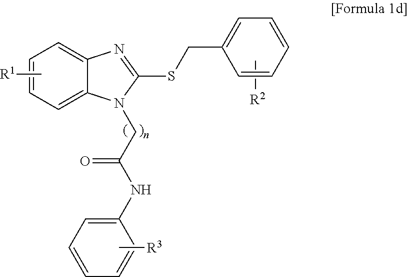
(wherein each of R¹, R², R³, and n is the same as defined in claim 1).

7. The compound according to claim 1, which is a compound selected from:
2-(2-(2,5-dimethoxyphenyl)-1H-benzo[d]imidazol-1-yl)-N-(5-isopropyl-2-methylphenyl)acetamide,
N-(5-(biphenyl-4-yloxy)-2-fluorophenyl)-2-(2-(2,5-dimethoxyphenyl)-1H-benzo[d]imidazol-1-yl)acetamide,
N-(3,5-dichlorophenyl)-2-(2-(2,5-dimethoxyphenyl)-1H-benzo[d]imidazole-1-yl)acetamide,
N-(5-isopropyl-2-methylphenyl)-(2-phenyl-1H-benzo[d]imidazol-1-yl)acetamide,
N-(3,5-dichlorophenyl)-2-(2-(2,5-dichlorophenyl)-1H-benzo[d]imidazol-1-yl)acetamide,
N-(3,5-di-tert-butylphenyl)-2-(2-(2,5-dichlorophenyl)-1H-benzo[d]imidazol-1-yl)acetamide,
N-(2-tert-butyl-6-methylphenyl)-2-(2-(2,5-dichlorophenyl)-1H-benzo[d]imidazol-1-yl)acetamide,
2-(2-benzyl-1H-benzo[d]imidazol-1-yl)-N-(5-isopropyl-2-methylphenyl)acetamide,
2-(2-benzyl-1H-benzo[d]imidazol-1-yl)-N-(5-biphenyl-4-yloxy)-2-fluoro phenyl)acetamide,
2-(2-benzyl-1H-benzo[d]imidazol-1-yl)-N-(3,5-dichlorophenyl)acetamide,
2-(2-benzyl-1H-benzo[d]imidazol-1-yl)-N-(3,5-tert-butylpenyl)acetamide,
2-(2-benzyl-1H-benzo[d]imidazol-1-yl)-N-(2-tert-butyl-6-methylphenyl)acetamide,
N-(3,5-di-tert-butylphenyl)-2-(2-phenyl-1H-benzo[d]imidazol-1-yl)acetamide,
N-(5-isopropyl-2-methylphenyl)-2-(2-phenyl-1H-benzo[d]imidazol-1-yl)acetamide,
N-(3,5-dichlorophenyl)-2-(2-phenyl-1H-benzo[d]imidazol-1-yl)acetamide,
N-(2-tert-butyl-6-methylphenyl)-2-(2-phenyl-1H-benzo[d]imidazol-1-yl)acetamide,
N-(3,5-di-tert-butylphenyl)-2-(2-(2,5-dimethoxybenzyl)-1H-benzo[d]imidazol-1-yl)acetamide,
N-(3,5-dichlorophenyl)-2-(2-(2,5-dimethoxybenzyl)-1H-benzo[d]imidazol-1-yl)acetamide,
2-(2-(2,5-dimethoxybenzyl)-1H-benzo[d]imidazol-1-yl)-N-(5-isopropyl-2-methylphenyl)acetamide,
N-(3,5-di-tert-butylphenyl)-2-(2-(2,6-dichlorobenzyl)-1H-benzo[d]imidazol-1-yl)acetamide,
2-(2-(3,4-dichlorobenzyl)-1H-benzo[d]imidazol-1-yl)-N-(5-isopropyl-2-methylphenyl)acetamide,
2-(5-chloro-2-(2,6-dichlorobenzyl)-1H-benzo[d]imidazol-1-yl)-N-(5-isopropyl-2-methylphenyl)acetamide,
2-(2-(2,6-dichlorobenzyl)-1H-benzo[d]imidazol-1-yl)-N-(5-isopropyl-2-methyl)acetamide,
N-(3,5-di-tert-butylphenyl)-2-(2-(3,4-dichlorobenzyl)-1H-benzo[d]imidazol-1-yl)acetamide,
2-(6-chloro-2-(2,5-dichlorophenyl)-1H-benzo[d]imidazol-1-yl)-N-(5-isopropyl-2-methylphenyl)acetamide,
2-(5-chloro-2-(2,5-dichlorophenyl)-1H-benzo[d]imidazol-1-yl)-N-(5-isopropyl-2-methylphenyl)acetamide,
2-(5-chloro-2-(2,5-dichlorophenyl)-1H-benzo[d]imidazol-1-yl)-N-(3,5-dichlorophenyl)acetamide,
2-(6-chloro-2-(2,5-dichlorophenyl)-1H-benzo[d]imidazol-1-yl)-N-(3,5-dichlorophenyl)acetamide,
2-(2-(3,4-dichlorobenzyl)-1H-benzo[d]imidazol-1-yl)-N-(3,5-dichlorophenyl)acetamide,
2-(6-chloro-2-(2,5-dichlorophenyl)-1H-benzo[d]imidazol-1-yl)-N-(3,5-di-tert-butylphenyl)acetamide,
2-(5-chloro-2-(2,5-dichlorophenyl)-1H-benzo[d]imidazol-1-yl)-N-(3,5-di-tert-butylphenyl)acetamide,
4-((2-(2,6-dichlorobenzyl)-1H-benzo[d]imidazol-1-yl)methyl)-N-((1-ethylpyrrolidin-2-yl)methyl)benzamide,
4-((2-(2,6-dichlorobenzyl)-1H-benzo[d]imidazol-1-yl)methyl)-N-((1-methylpiperidin-4-yl)methyl)benzamide,
4-((2-(2,6-dichlorobenzyl)-1H-benzo[d]imidazol-1-yl)methyl)-N-(3-(dimethylamino)propyl)benzamide,
4-((2-(2,6-dichlorobenzyl)-1H-benzo[d]imidazol-1-yl)methyl)-N-(3-(pyrrolidin-1-yl)propyl)benzamide,
4-((2-(2,6-dichlorobenzyl)-1H-benzo[d]imidazol-1-yl)methyl)-N-(3-(4-methylpiperazin-1-yl)propyl)benzamide,
4-((2-(2,6-dichlorobenzyl)-1H-benzo[d]imidazol-1-yl)methyl)-N-(3-(piperidin-1-yl)propyl)benzamide,
4-((2-(2,6-dichlorobenzyl)-1H-benzo[d]imidazol-1-yl)methyl)-N-(3-(piperidin-1-yl)ethyl)benzamide,
4-((2-(2,6-dichlorobenzyl)-1H-benzo[d]imidazol-1-yl)methyl)-N-(3-morpholinopropyl)benzamide,
2-(4-chloro-2-(2,6-dichlorobenzyl)-1H-benzo[d]imidazol-1-yl)-N-(5-isopropyl-2-methylphenyl)acetamide,
N-((1-ethylpyrrolidin-2-yl)methyl)-4-((2-(2-(5-isopropyl-2-methylphenylamino)₂-oxoethyl)-1H-benzo[d]imidazol-1-yl)methyl)benzamide,
N-(3-(dimethylamino)propyl)-4-((2-(2-(5-isopropyl-2-methylphenylamino)-2-oxoethyl)-1H-benzo[d]imidazol-1-yl)methyl)benzamide,
N-((1-ethylpyrrolidin-2-yl)methyl)-4-((1-(2-(5-isopropyl-2-methylphenylamino)-2-oxoethyl)-1H-benzo[d]imidazole-2-ylthio)methyl)benzamide,
4-((2-(2-(5-isopropyl-2-methylphenylamino)-2-oxoethyl)-1H-benzo[d]imidazol-1-yl)methyl)-N-(3-piperidin-1-yl)propyl)benzamide,
N-(3-(diethylamino)propyl)-4-((1-(2-(5-isopropyl-2-methylphenylamino)-2-oxoethyl)-1H-benzo[d]imidazole-2-ylthio)methyl)benzamide, and
4-((1-(2-(5-isopropyl-2-methylphenylamino)-2-oxoethyl)-1H-benzo[d]imidazol-2-ylthio)methyl)-N-(3-piepridin-1-yl)propyl)benzamide.

8. A pharmaceutical composition for treating a disease selected from Alzheimer's disease, Parkinson's disease, Huntington's disease, ischemic brain diseases, diabetes and schizophrenia, which comprises the compound as defined in claim 1 as an active ingredient.

9. A pharmaceutical composition for treating a disease selected from Alzheimer's disease, Parkinson's disease, Huntington's disease, ischemic brain diseases, diabetes and schizophrenia, which comprises the compound as defined in claim 2 as an active ingredient.

10. A pharmaceutical composition for treating a disease selected from Alzheimer's disease, Parkinson's disease, Huntington's disease, ischemic brain diseases, diabetes and schizophrenia, which comprises the compound as defined in claim 3 as an active ingredient.

11. A pharmaceutical composition for treating a disease selected from Alzheimer's disease, Parkinson's disease, Huntington's disease, ischemic brain diseases, diabetes and schizophrenia, which comprises the compound as defined in claim 4 as an active ingredient.

12. A pharmaceutical composition for treating a disease selected from Alzheimer's disease, Parkinson's disease, Huntington's disease, ischemic brain diseases, diabetes and schizophrenia, which comprises the compound as defined in claim 5 as an active ingredient.

13. A pharmaceutical composition for treating a disease selected from Alzheimer's disease, Parkinson's disease, Huntington's disease, ischemic brain diseases, diabetes and schizophrenia, which comprises the compound as defined in claim 6 as an active ingredient.

14. A pharmaceutical composition for treating a disease selected from Alzheimer's disease, Parkinson's disease, Huntington's disease, ischemic brain diseases, diabetes and schizophrenia, which comprises the compound as defined in claim 7 as an active ingredient.
